(19) United States
(12) Patent Application Publication
(10) Pub. No.: US 2022/0016364 A1
(43) Pub. Date: Jan. 20, 2022

(54) ADAPTED E-CIGARETTE WITH AUTHENTICATION FOR NICOTINE DELIVERY

(71) Applicant: SMOKELESS.WORLD GMBH, Grünwald (DE)

(72) Inventors: Daniel KILGER, München (DE); Patrick MÜLLER, Heiningen (DE); Michael SEDLMEIER, München (DE)

(21) Appl. No.: 17/295,749

(22) PCT Filed: Nov. 18, 2019
(86) PCT No.: PCT/EP2019/081663

§ 371 Date: May 20, 2021

(30) Foreign Application Priority Data
Nov. 22, 2018 (EP) 18207886.5
Mar. 29, 2019 (EP) 19166366.5

(51) Int. Cl. A61M 15/00; G16H 20/10; A24F 40/30; A24F 40/10; A24F 40/42; A24F 40/60; A24F 40/53; A24F 40/65

(52) U.S. Cl. CPC A61M 15/008; G16H 20/10; A24F 40/30; A24F 40/10; A24F 40/42; A61M 2205/276; A24F 40/53; A24F 40/65; A61M 2205/52; A61M 2205/502; A24F 40/60

(57) ABSTRACT

The present invention is directed to a system comprising a mobile inhaler (1) that is configured to be simultaneously connected to at least one or a plurality of liquid containers (17) and to deliver an inhaling substance (10), wherein the inhaling substance comprises at least one of an amount of a first component (11) and an amount of a second component (12). The mobile inhaler further comprises at least one or a plurality of connector(s) (80) configured for connection of the mobile inhaler to the at least one or the plurality of liquid containers and for intaking of at least a portion of each of the liquid container(s)'s content(s). The system comprises furthermore the at least one or the plurality of liquid containers, wherein the at least one or the plurality of liquid containers comprises a plurality of reservoirs, wherein each reservoir comprises a component of the inhaling substance and wherein the reservoirs each comprise mutually different components of the inhaling substance. The present invention is furthermore directed to a method for operating the system, comprising delivering the inhaling substance by the mobile inhaler, wherein the mobile inhaler delivers at least the first component of the inhaling substance according to a set of conditions. Said set of conditions regulates at least one of a quantity and a general delivery of at least one of the first component and the inhaling substance.

### FIELD

The present invention relates to an inhaler, such as a portable inhaler or more specifically to an e-cigarette, for automatically dosing at least one component, such as nicotine or Cannabis, in an inhaling composition with more than one component, if a certain pre-defined condition or a set thereof is met.

### BACKGROUND

According to Wikipedia of October 2018, an electronic cigarette or e-cigarette is a handheld electronic device that simulates the feeling of smoking. It works by heating a liquid to generate an aerosol, commonly called a “vapor”, that the user inhales. Using e-cigarettes is commonly referred to as vaping. The liquid in the e-cigarette, called e-liquid, or e-juice, is usually made of nicotine, propylene glycol, glycerine, and flavorings. Not all e-liquids contain nicotine.

E-cigarettes can create an aerosol, commonly called vapor. Its exact composition varies. The majority of toxic chemicals found in tobacco smoke are absent in e-cigarette aerosol. Those present are mostly below 1% of the corresponding levels in tobacco smoke. The aerosol can contain toxicants and traces of heavy metals at levels permissible in inhalation medicines, and potentially harmful chemicals not found in tobacco smoke at concentrations permissible by workplace safety standards.

The modern e-cigarette was invented in 2003 by Chinese pharmacist Mr. Hon Lik, and as of 2018 most e-cigarettes are made in China. Since they were first sold in 2004 their global use has risen exponentially. In the United States and the United Kingdom their use is widespread. Reasons for using e-cigarettes involve trying to quit smoking, reduce risk, or save money, though some use them recreationally. As of 2014, the majority of users still smoke tobacco. There are concerns that dual use of tobacco products and e-cigarettes may “delay or deter quitting”. About 60% of UK users are smokers and roughly 40% are ex-smokers. In the UK use among never-smokers was negligible.

Electronic cigarettes are also known as e-cigarettes, e-cigs, EC, electronic nicotine delivery systems (ENDS) or electronic non-nicotine delivery systems (ENNDS), electronic smoking devices (ESDs), personal vaporizers, or PVs. They are handheld devices, often made to look like conventional cigarettes, and used in a similar way.

There are three main types of e-cigarettes: cigalikes, looking like cigarettes; eGos, bigger than cigalikes with refillable liquid tanks; and mods, assembled from basic parts or by altering existing products. As the e-cigarette industry continues to evolve, new products are quickly developed and brought to market. First generation e-cigarettes tend to look like tobacco cigarettes and so are called “cigalikes”. Most cigalikes look like cigarettes but there is some variation in size. A traditional cigarette is smooth and light while a cigalike is rigid and slightly heavier. Second generation devices are larger overall and look less like tobacco cigarettes. Third generation devices include mechanical mods and variable voltage devices. The fourth generation includes Sub ohm tanks and temperature control devices. The power source is the biggest component of an e-cigarette, which is frequently a rechargeable lithium-ion battery.

The main components of an e-cigarette are a mouthpiece, a cartridge (tank), a heating element/atomizer, a microprocessor, a battery, and possibly an LED light on the end. The only exception to this are mechanical e-cigarettes (mods) which contain no electronics; the circuit is closed by a mechanical action switch. An atomizer comprises a small heating element, or coil, that vaporizes e-liquid and wicking material that draws liquid onto the coil. When the user pushes a button, or (in some variations) activates a pressure sensor by inhaling, the heating element atomizes the liquid solution. The e-liquid reaches a temperature of roughly 100-250° C. within a chamber to create an aerosolized vapor, which the user then inhales, rather than cigarette smoke. The aerosol provides a flavor and feel similar to tobacco smoking.

E-liquid or juice are names for the flavored solution that goes inside the e-cigarette. An aerosol, or vapor, is produced by heating the e-liquid. Irish public health discussions refer to NMNDS (“non-medicinal nicotine delivery systems”). When the FDA commissioned their 2018 report on ENDS which they label as a Tobacco Product, the authors chose to use the term e-cigarettes for some using e-juice without nicotine.

E-liquid is the mixture used in vapor products such as e-cigarettes and generally consists of propylene glycol, glycerin, water, nicotine, and flavorings. While the ingredients vary the liquid typically contains 95% propylene glycol and glycerin. There are many e-liquids manufacturers in the US and worldwide, and upwards of 8,000 flavors. Industry standards have been created and published by the American E-liquid Manufacturing Standards Association (AEMSA).

Between their introduction to the market in 2004 and approximately 2015, global usage of e-cigarettes rose exponentially. By 2013, there were several million users globally.

Awareness and use of e-cigarettes greatly increased in a relatively short period of time. Growth rates in the US and UK slowed in 2015, although use is still increasing.

Most users have a history of smoking regular cigarettes. At least 52% of smokers or ex-smokers have vaped. Of smokers who have, one British study reported that less than 15% became everyday e-cigarette users. One United States survey of e-cigarette users conducted from 2011-2012 found that only 1% of respondents used liquid without nicotine.

E-cigarettes may be used with other substances and cartridges can potentially be filled with e-liquid containing substances other than nicotine, thus serving as a new way to deliver other psychoactive drugs, for example Cannabis.

Cannabis, also known as marijuana among other names, is a psychoactive drug from the Cannabis plant or synthesized used for medical or recreational purposes. The main psychoactive part of Cannabis is tetrahydrocannabinol, one of 483 known compounds in the plant, including at least 65 other cannabinoids. Cannabis can be used by smoking, vaporizing, within food, or as an extract. The term Cannabis is intended to cover THC, CBD or cannabinoids, terpenes etc.

The emergence of e-cigarettes has given Cannabis smokers a new method of inhaling cannabinoids. E-cigarettes, also known as vape pens, cartridges and pens, differ from traditional marijuana cigarettes in several respects. It is assumed that vaporizing cannabinoids at lower temperatures is safer because it produces smaller amounts of toxic substances than the hot combustion of a marijuana cigarette. Recreational Cannabis users can discreetly “vape” deodorized Cannabis extracts with minimal annoyance to the people around them and less chance of detection, known as “stealth vaping”. While Cannabis is not readily soluble in the liquid used for e-cigs, recipes containing synthetic cannabinoids which are soluble may be found.

The legal use and distribution of e-cigarettes with juices containing nicotine as well as nicotine-free juices as well as the consumption of cannabinoids depends on the local legislation. In Germany, selling, offering and allowing to use e-cigarettes to minors is not compliant to the Youth Protection Act, according to Wikipedia of January 2019. The German Youth Protection Act does not regulate the use of e-cigarettes in private, especially under parental supervision. Whether restrictions on the sale to minors, use in public or the like applies depends on legislation in the regarding country or state and may furthermore depend on third parties, such as a transportation authority (partially) banning or not banning the use of electronic cigarettes in their vehicles and stations.

EP 2 608 686 B1 is directed to an inhalation device comprising two cassettes with two dispensing devices with each being associated to one ingredient. The inhalation device can estimate the number of cigarette equivalents remaining based on an estimation of the remaining ingredient and display this by means of a display.

Furthermore, the U.S. Pat. No. 7,028,693 B2 provides a procedure for the weaning of smoking articles, in which a cigarette dispenser is provided to control the dispensing of cigarettes out of a cigarette pack.

WO 2015 150 699 A1 describes a device for setting into a portable terminal device a quantity of an active substance inhaled by a user. For the mentioned adjustment of the amount of active substance the volume of steam inhaled by the user is measured.

EP 2 276 360 B1 relates to an electrically heated smoking system for receiving an aerosol-forming substrate.

### SUMMARY

It is the object of the present invention to provide an improved or alternative mobile inhaler, particularly an e-cigarette, and a respective method for an improved or alternative way of inhaling from a mobile inhaler, particularly from an e-cigarette.

It is another optional object to provide a mobile inhaler, particularly an e-cigarette, and a respective method of inhaling that allow a more flexible way of inhaling and a respective method of inhaling.

It is another optional object of the present invention to provide a mobile inhaler, particularly an e-cigarette, and a respective method of inhaling that allow to automatically and to flexibly mix ingredients in an inhaling composition and/or control one or more components from the composition.

It is another optional object of the present invention to provide a mobile inhaler, particularly an e-cigarette, and a respective method of inhaling that allow to automatically regulate harmful, addictive and/or psychoactive components of the inhaling composition over time.

It is another optional object of the present invention to provide a mobile inhaler, particularly an e-cigarette and a respective method, to verify conditions before activating or enabling the mobile inhaler.

It is still another optional objective of the present invention to provide a mobile inhaler and a respective method to impose restrictions regarding the use of liquid containers from which an inhaling substance or components thereof are delivered.

It is another optional object to provide an assembly of an e-cigarette that can be controlled in an optimized manner and to inter alia make use of other components.

It is a still further optional object to provide a system comprising an e-cigarette and a handheld device enabling better handling by a user and further computing abilities and power.

Each of the above objects can be attained with the subject matter of the present invention as recited in the claims, embodiments and/or description.

If in this document, there are references towards a wired or wireless connection, wired or wireless data transfer or a link between two devices that is configured to exchange data, then the person skilled in the art will understand that this is not intended to limit the connection/data transfer method/link configured to exchange data to a direct or indirect connection between the devices that are connected, linked or exchanging data or configured to do so. This does obviously not apply in cases where this document states the opposite or specifies a direct or indirect connection.

The present invention is directed to a system comprising a mobile inhaler, which can also be called a mobile inhaling device. It can be an e-cigarette.

The mobile inhaler is configured to be connected to at least one or a plurality of liquid containers. The system is furthermore configured to deliver an inhaling substance that comprises at least one of an amount of a first component of the inhaling substance and an amount of a second component of the inhaling substance. The inhaling substance is a composition that is delivered to a user for an inhalation. The inhaling substance can be an inhaling mixture, such as an aerosol for inhalation or a spray for inhalation. The inhaling substance comprises at least one of the first and the second component. This inhaling substance can be any mixture of those components, comprising the mixtures that consist of only one of the components. In this disclosure, a component is understood as a composition that is at least partially a fluid and that may comprise several chemical substances or compounds.

In some embodiments, the mobile inhaler can comprise furthermore at least one or a plurality of connectors. This connector or those connectors are configured for connection of the mobile inhaler to the at least one or the plurality of liquid containers and for intake of at least a portion of each of the liquid container(s)'s content(s), such as the first and the second component of the inhaling substance or parts thereof, e.g. if a portion of the first and/or the second component of the inhaling substance remains in the liquid container(s). Those connectors or this connector can be connectors that are liquid tight.

The system can furthermore comprise the one or the plurality of liquid containers. The liquid container(s) can be permanently or non-permanently attached to the mobile inhaler. The at least one or the plurality of liquid containers can comprise a plurality of reservoirs, and each reservoir can comprise a component of the inhaling substance. That is, the system can for example comprise one liquid container that is comprising a plurality of reservoirs, or two liquid containers that each comprise one or more reservoirs, as even in the case where each of the two liquid containers comprises one reservoir, the two liquid containers as concrete example of at least one or the plurality of liquid containers then comprise a plurality of reservoirs. A reservoir is any container that is configured to comprise one liquid, whereas it can furthermore comprise another gas, such as air.

In some embodiments, the reservoirs can each comprise mutually different components of the inhaling substance. That is, the molar concentration of a least one chemical substance of which the components are composed can be different between each pair of components of the inhaling substance, wherein this chemical substance which has a differing molar concentration does not need to be the same for each pair of components of the inhaling substance.

The components comprised by each pair of reservoirs can differ in the way that the molar concentration of at least one chemical substance differs at least by a factor of ten between the two components in the pair. For more than two reservoirs and components, the substance whose molar concentration is different by at least a factor of ten for each pair can be different for each pair of components. As an example, in the case of three components (C1, C2 and C3), for a first pair of components (C1 and C2), an exemplary chemical substance A can be present in one of C1 and C2 with a molar concentration that is at least ten times higher than in the other component. For another pair of components (C2 and C3), the substance whose molar concentration differs by at least a factor of ten can be another substance B, but it can also be the substance A. For a last pair of components (C3 and C1), the chemical substance whose molar concentration differs by at least a factor of ten can then be any of the substances A and B or a third substance, such as a substance C.

The first component of the inhaling substance comprises preferably nicotine, a nicotine derivate or another psychoactive substance such as Cannabis.

In some embodiments, the components of the inhaling substance that can be stored in the liquid container(s) can be at least partially liquids. The reservoirs can each comprise one of the components, whereas they can also each comprise air, a gaseous phase of the respective component and/or another gas or liquid. The components can nevertheless be substantially liquid under standard conditions.

In some embodiments, each liquid container can comprise at least one or a plurality of connectors configured for connection of the respective liquid container to the mobile inhaler. The connector can also be configured to connect the liquid container indirectly to the mobile inhaler, such as by connecting one liquid container I to another liquid container II, wherein liquid container II is then connected to the mobile inhaler. The connection may comprise a further connecting piece in between said mobile inhaler and the respective liquid container. The connecting piece may also comprise an element configured for combining the connections of different liquid containers to said mobile inhaler.

In some optional embodiments, at least one of the at least one or the plurality of liquid containers is at least one liquid container that comprises the plurality of reservoirs. In such embodiments, the system can for example comprise one liquid container that comprises two reservoirs.

In other optional embodiments, the at least one or the plurality of liquid containers is a plurality of liquid containers that comprises a plurality of reservoirs. In such embodiments, the system can for example comprise two liquid containers that each comprise one reservoir.

In some embodiments, the system can comprise a dosing component that is configured to perform at least the dosing of at least a component of the inhaling substance. In such embodiments, the system can comprise a dosing component that is configured to perform the dosing of at least one of the first component and the second component of the inhaling substance. The mobile inhaler can comprise at least a portion of the dosing component or the dosing component. At least one of the at least one liquid containers can comprise at least a portion of the dosing component or the dosing component. In some embodiments, each liquid container can comprise a dosing component.

In such embodiments where the system comprises a dosing component that is configured to perform the dosing of at least one of the first and the second component of the inhaling substance, the dosing component can be configured to perform the dosing of at least the first component and the second component of the inhaling substance independently from each other. In those embodiments, the dosing component can comprise two dosing mechanisms that perform the dosing of at least the first component and at least the second component respectively.

In some embodiments, the system comprises furthermore a computing component. In such embodiments, the mobile inhaler can comprise at least a portion of the computing component. That is for example, the mobile inhaler can comprise at least one computing unit and the computing component comprises this at least one computing unit.

The computing component can be any element that is configured to perform calculations and that is furthermore configured to be programmed, such as a micro-controller.

The computing component can at least be configured to perform calculations that are necessary for at least one of a dosing and a delivery of at least one of the inhaling substance, the first component and the second component of the inhaling substance.

The computing component can optionally comprise a plurality of computing components. These computing components can be distributed, such as that for example one of the at least one or the plurality of liquid containers comprises one computing component and the mobile inhaler comprises a computing component.

The computing component can be an integrated circuit.

The computing component can be at least one of a micro-controller, a micro-processor, an ASIC, an FPGA or a CPLD, especially in the case where the computing component comprises two integrated circuits that are configured to build a computing system together.

In some embodiments, the system can comprise furthermore a data storage component that is at least configured to store data. At least one of the mobile inhaler and at least one of the liquid container or liquid containers can comprise at least a portion of the data storage component. The data storage component can also be a memory component.

The data storage component can be an electronic storage element, such as a memory chip, a microcontroller, a microprocessor or at least a portion thereof, or it can be an integrated circuit, a flash memory component, a RAM component, a read-only memory component or a hard disk. In particular for the storage of binary information, the data storage component can be an electrical or electronical component that is destroyed, such as a fuse that is burnt or a microcontroller or an electronical circuit that is short-circuited or a mechanical element that is deformed, removed or added, such as a metal that is bent by mechanical, thermal or magnetic forces or a piece of metal that links two electric contacts and that is removed or added. The data storage component can also comprise multiple elements selected from the list of elements that can be a data storage component.

In some embodiments, the computing component can at least be configured to perform at least a part of a calculation for regulating the amount of at least the first component of the inhaling substance that is delivered over time. This calculation can be based on a pre-defined mathematical or logical pattern or function or a pre-defined rule-set, such as an adaption of the amount of the first component when certain criteria, e.g. an action of a user, are matched. The calculation can also be based on a pattern based on data, wherein the data is at least one of measured, generated and recorded by the mobile inhaler. Apart from those aforementioned options, the calculation can also be based on a mathematical model or function based on data that is at least measured, generated and/or recorded by the mobile inhaler, or the calculation can be based on a machine learning or artificial intelligence model, such as a reinforcement learning model that was trained with data sets of the consumption of users and the delivery of components or chemical substances by inhalation. This disclosure considers neural networks to be a part of machine learning and artificial intelligence models. The calculation for the regulation of the amount of at least the first component that is delivered over time can also be based on further data that is input from other sources, such as an input from trained medical personnel. It can furthermore be based on a combination of the aforementioned options.

In some embodiments, the system can comprise at least one or a plurality of information input elements. The information input element can be a localisation component, such as a component to determine the mobile inhaler's position based on a regional or global navigation satellite system. The information input element can also be a sound sensor, such as a microphone. The information input element can be an interaction element for a user or a third party that is configured to signal to the mobile inhaler to deliver or to deliver at all the inhaling substance or a component thereof. The interaction element can for example be configured to trigger an extra-delivery of the component of the inhaling substance that comprises nicotine or another psychoactive substance. The interaction element can also be configured to signal to the mobile inhaler to raise, to lower and/or to adapt at least the delivered amount of the inhaling substance or of a component thereof. The interaction element can be an element such as a physical knob or button or a button on a touch-sensitive display. The button on a touch-sensitive display can be a button in a software running on a mobile device that is connected to the mobile inhaler or a control at a medical device or software connected to the mobile inhaler. The interaction element does not need to be configured for an interaction with the user, as stated above, it can also be configured for an interaction with a third party, such as medical personnel adapting the delivery of a psychoactive substance for a pain treatment, or parents authorizing the delivery of nicotine to a minor in their custody, depending on the applicable youth protection act.

The information input element can also be one or a plurality of sensing devices to sense the user's physiological state, such as a sensing device to sense the user's blood pressure, the user's pulse, (an) indicator(s) for emotional stress, a potential medication of the user, a potential intoxication of the user, the user's transpiration or the presence of diseases. It can furthermore be a clock, a timer and/or a wearable biological or medical sensor. The information input element can also be a database comprising at least labels for positions, such as “bar”, “station”, “place with smoking ban” or “place with vaping ban”, wherein those labels can be used to determine whether a general or particular use of the mobile inhaler (e.g. with or without the delivery of nicotine/Cannabis as a first component) is prohibited.

The information input element can also be a device configured to sense a measure related to the step of delivering at least one of a component of the inhaling substance and the inhaling substance, such as a pressure sensing device, a sound sensing device, an airflow sensing device or a signal from the user to perform the delivery. The information input element can also be a finger print sensor, and/or a tactile sensor.

In some embodiments, the mobile inhaler can be configured for connection to at least one, a plurality or all of the at least one or a plurality of information input elements. This connection can be direct or indirect. In this case, the interaction element for a user or a third party can be for example a button in a software running on a mobile end user device that is connected to the mobile inhaler or a control at a medical device or a medical software that is connected to the mobile inhaler. An indirect connection can be an advantage when a third party that is signalling to the mobile inhaler is at a remote location, for example medical personnel treating an addict that does not permanently stay in a care facility, or when parents authorise the use of nicotine or another psychoactive substance to their children, if that complies with the applicable youth protection act.

In some embodiments, the mobile inhaler can comprise at least one, a plurality or all of the at least one or a plurality of information input elements.

In some embodiments, the system can comprise furthermore an interface suitable to connect the system to at least one of a computer device, an integrated circuit and a data storage device by at least one of a wired and a wireless connection. The interface can also be an interface device, such as a USB-connector, a device configured to connect the mobile inhaler to a wireless LAN or a device to connect the mobile inhaler to another bus, such as a CAN-bus.

The interface suitable to connect the system to at least one of a computer device, an integrated circuit and a data storage device can furthermore be configured to connect the system to at least one of a computer device, an integrated circuit and a data storage device.

The interface can be an interface for wired communication, such as a USB-interface or an interface configured to physically connect the mobile inhaler to at least one of a dongle, a SIM-card, an SD-card, a chip card, a magnetic stripe and any other integrated circuit element.

The interface can also be an interface for wireless communication, such as an interface for radio communication, such as interfaces to communicate with RFID-chips or communication systems marketed as Bluetooth™ or Wi-Fi™, NFC communication, communication via mobile internet or via cellular network services.

The interface can furthermore be configured for direct or indirect communication. Direct wired communication can be a communication that directly links the communicating devices, such as a USB connection or a radio connection between two devices. Indirect communication can be communication that links the communicating devices via an at least one intermediate device, such as a connection between to devices that are connected to a WLAN or a LAN, wherein usually at least a router acts as intermediate device, or a connection via the internet, that usually comprises a plurality of intermediate devices.

The system can comprise a document checking component configured to verify and/or check a property provided by at least one of an official identity document, a mean of payment and another document to confirm the holder's identity. The official identity document can be an official identity document such as an ID-card, a passport, an alien registration card or another official document issued for the purpose of the holder's identification. A mean of payment can be any mean of payment, such as a credit card, another mean of cashless payment such as a mobile phone payment system, but it can also be any other medium of exchange, comprising guarantees of financial service providers such as those made by online payment service providers. Another document to confirm the holder's identity can be for example a driving license, a health insurance card, which would be particularly useful if the health insurance card stored medical data that are relevant for delivering the inhaling substance or one of its components, but it could also be any other document suitable to confirm the holder's identity, such as a card or document issued by the supplier of the mobile inhaler.

The document checking component can be a computing device with an appropriate sensing device and an appropriate software, and the computing device can be connected to the mobile inhaler at least indirectly.

The computing device connected to the mobile inhaler can be a mobile device, such as portable personal computer or a smartphone, or an end user device, that furthermore comprises an appropriate application or an appropriate set of applications.

The application or at least one application of the set of applications on the mobile device can be configured to communicate data to a remote server in order to verify and/or check a property provided by at least one of an identity document and at least one mean of payment. The remote server can be a server to which the mobile device is connected via the internet.

The mobile inhaler can comprise furthermore a connection configured to transfer data to/from an input interface element, wherein the input interface element is configured to receive data input from a user, such as input of a PIN, a password or voice.

The system can furthermore comprise the input interface element that is configured to receive data input from a user.

The mobile inhaler can comprise the input interface element that is configured to receive data input from a user.

The input interface element can be at least one of an interface of a mobile device and an interface of a computer device, and the respective device can be connected to the mobile inhaler at least at one point in time. The respective device can be an end user computer device as defined later on in this disclosure.

The mobile inhaler can comprise a connection component to an output interface component. The output interface component can be a user interface or a component configured to implement a user interface.

The system can comprise the output interface component.

In such embodiments, the system can comprise the output interface component.

The output interface component can be configured to display information relating to the mobile inhaler, a delivery of the inhaling substance and/or an inhalation, information relating to a recording and/or a duration of an inhalation, a summary or an analysis thereof or information relating to at least one liquid container that is, was or can be connected to the mobile inhaler. The displaying information in this context also comprise playing audio comprising the information, or communication the information to the user by other media, such as via a vibration. The information relating to at least one liquid container that can be connected to the mobile inhaler may also comprise advertisement.

The output interface component can be at least one of a visual interface device, such as an LED, an LED-array, a screen or a projector, an acoustic output device, such as a speaker, a buzzer or another device configured to play audio data, and a haptic element, such as a vibrating element.

The output interface component can be at least one of a computer device and a display device that can be connected to a mobile inhaler, such as a smartphone, a personal computer, a printer, a screen or a virtual reality headset. A display device can be connected to the mobile inhaler with a wireless connection configured to transfer data, such as via WLAN or wireless mobile internet.

In some embodiments, the mobile inhaler can comprise furthermore a connection component and this connection component can be configured to connect the mobile inhaler at least to a controlling control at least indirectly and at least at one point in time, preferably at least at some points in time.

The system can furthermore comprise said controlling control.

The controlling control can be configured to at least influence the delivery of at least the first component of the inhaling substance.

The controlling control can be implemented in software and the controlling control can furthermore at least partially be running on at least one of a smart handheld device, a remote server, a cloud computing system, a medical device, and another computer or computer system carrying out the task of running the controlling control.

In some embodiments, the mobile inhaler can comprise furthermore at least one or a plurality of delivering restriction(s) and at least one of the liquid container(s) can comprise at least one or a plurality of matching restriction element(s). Each of the at least one delivering restrictions is a feature configured to prevent delivering at least one component of the inhaling substance by the mobile inhaler from a liquid container without the respective restriction element(s).

In such embodiments, the mobile inhaler can be configured to deliver at least one component of an inhaling substance only if the at least one or the plurality of delivering restriction(s) are matched by at least a part of the restriction element(s) of at least one of the respective liquid container(s), and/or if there is another authorization, such as an authorization by a third party that disposes of a sufficient authentication, e.g. a pharmacist who produced one or more components of the inhaling substance according a medical prescription. In a situation where the maximum concentration of a substance such as nicotine or Cannabis in a reservoir is known, a regulation of the amount of the delivered psychoactive substance can be made more reliable by restricting the reservoir(s) or liquid container(s) that comprise the second component of the inhaling substance.

In such an embodiment, the mobile inhaler can furthermore be configured to deliver at least one component of the inhaling substance only if the delivering restriction(s) are matched by at least a part of the restriction element(s) of at least one of the respective liquid container(s), wherein furthermore at least one of the delivering restriction(s) is specific to a to a subset of the plurality of reservoirs and the liquid container(s) comprising this subset, and/or if there is another authorization. This subset of reservoir can for example be characterized by that they do not comprise any component that comprises a psychoactive component.

In such an embodiment, the mobile inhaler can furthermore be configured to deliver at least one component of the inhaling substance only if the at least one or the plurality of delivering restriction(s) are matched by at least a part of the at least one or the plurality of restriction element(s) of all liquid container(s) that are connected to the mobile inhaler and/or if there is another authorization.

At least one of the at least one restriction element(s) can comprise the shape of at least one liquid container.

At least one of the at least one of the mobile inhaler's delivering restriction(s) can comprise an element that limits the liquid container or liquid containers that can be connected to the mobile inhaler at least based on their shape.

In such embodiments, this element can comprise a cavity in the mobile inhaler that is configured to accommodate at least one of the liquid container(s) with one or a plurality of restriction elements, such as a cavity with a limited width and/or a limited length or a cavity with a particular geometry of at least a portion of the cavity, such as a geometry of at least a portion of the cavity selected from a cone-shape, a paraboloid of revolution, a hyperboloid of revolution, a spheroid, a pyramidal shape, a cuboid shape and a freeform surface; and/or the geometry of the cavity comprising furthermore a bar, a screw thread, a bolt, a hole or an asymmetry.

At least one of the at least one restriction element(s) can comprise the connector of at least one of the fluid container(s) to at least one of the mobile inhaler's connector(s).

At least one of the mobile inhaler's delivering restriction(s) can comprise at least one of the mobile inhaler's connector(s).

In such embodiments, at least one of the mobile inhaler's delivering restriction(s) that comprises at least one of the mobile inhaler's connector(s) can comprise furthermore the shape of the at least one of the mobile inhaler's connector(s), such as a polygon shape, a round shape, a round shape with an irregularity, such as an indentation, or a splined shaft shape.

At least one of the mobile inhaler's delivering restriction(s) that comprise at least one of the mobile inhaler's connector(s) can furthermore comprise a connector configured to require a minimum pressure in at least one of the reservoir(s) in at least one of the liquid container(s). The minimum pressure can be an overpressure.

At least one restriction element of at least one of the liquid container(s) can comprise an electronic element configured at least for an identification of the respective liquid container. In such embodiments, the identification can be performed by wired communication between the mobile inhaler and the liquid container, such as by using a USB-interface or an interface configured to physically connect the mobile inhaler to at least one of a dongle, a SIM-card, an SD-card, a chip card, a magnetic stripe and any other integrated circuit element. The identification can also be performed by wireless communication between the mobile inhaler and the liquid container, such as by using an interface for radio communication, such as interfaces to communicate with RFID-chips or communication systems marketed as Bluetooth™ or Wi-Fi™, or NFC communication. The identification can also comprise a combination of wired and wireless communication interfaces.

At least one of the mobile inhaler's delivering restriction(s) can comprise an element configured to identify at least one liquid container by an electronic element that is mounted to the liquid container and that is configured at least for an identification of the liquid container by at least one of wired and wireless communication according to the preceding paragraph. The communication for which the electronic element and the mobile inhaler are configured can be at least one of direct and indirect communication. An advantage of direct communication can be the independence from supplementary devices. An advantage of indirect communication can be a greater flexibility, such as the possibility to integrate a server into the communication process. The server can for example be configured to verify a unique identification code of an liquid container and to replace such a code by a corresponding information, such as the result of the verifying and the content of the liquid container.

The identification in the context of an electronical element that is mounted to a liquid container can refer to identifying the individual liquid container, but it can also refer to identifying only a property of the liquid container, such as its content, its date of production (and therefore its age) or the manufacturing site or equipment. The electronic element configured to identify the liquid container(s) can comprise information that is configured to be necessary for an authentication of the result of the identification, such as the liquid container's identity.

The system can comprise at least two reservoirs in at least one liquid container, each reservoir comprising one component of the inhaling substance. It can furthermore comprise a dosing arrangement that is configured to automatically regulate the amount of the first component that is delivered over time according to a set of conditions. The dosing arrangement can comprise a dosing component according to any of the preceding descriptions that specify the dosing component. The set of conditions can comprise at least one or a plurality of conditions, wherein the set can furthermore comprise logical links of the results of the conditions, such as the logical conjunction, the logical disjunction or the negation of all or a part of the boolean results of some conditions or of the intermediate results obtained by the aforementioned conjunctions, disjunctions or negations.

The dosing arrangement can be configured to dose at least the first component in time intervals of at most 200 ms.

The dosing arrangement can also be configured to regulate the amount of the first component that is delivered over time. In such embodiments, the regulating of the amount of the first component that is delivered over time can be based on a pre-defined pattern or function, a pre-defined rule-set, and/or on a pattern based on data that is at least one of measured, generated and recorded by the mobile inhaler. It can also be based on a mathematical model or function based on data that is at least measured, generated and/or recorded by the mobile inhaler, a machine learning or artificial intelligence model and/or further data. Those possible bases of the regulating of the amount of the first component are understood as detailed above in the context of the computing component of the mobile inhaler.

The mobile inhaler can be a battery driven mobile device and more preferably an electronic cigarette or e-cigarette.

In embodiments wherein the system comprises a dosing arrangement, the dosing arrangement can comprise a first aerosol generator and a second aerosol generator. The first aerosol generator can be configured to vaporize the first component of the inhaling substance to a first aerosol, preferably so as to provide at least air as a carrier component together with the first aerosol. The second aerosol generator can be configured to vaporize the second component of an inhaling substance to a second aerosol, preferably so as to provide at least air as a carrier component together with the second aerosol. In this context, an aerosol is understood to be a mixture of one or more gases and another substance or another set of substances, preferably a liquid in fine droplets. A liquid that is vaporized and mixed into the gas(es) will also be considered an aerosol.

The mobile inhaler can furthermore comprise a mouth piece and a canal that is configured to supply air to the mouth piece wherein at least one of the first and second aerosol generator(s) are configured to deliver at least one of the first and second aerosols to the canal at least indirectly. The mouth piece is an element that is configured to be used for an inhalation of the inhaling substance by the user.

The dosing arrangement can furthermore comprise a controller that is configured to control at least one of the first and second aerosol generators. The controller can be identical to the system's computing component. It can be the system's computing component or a portion thereof. All technical considerations about suitable electronical components apply analogously.

The controller can be configured to at least control at least one of activations of the first and second aerosol generators and an amount of aerosol generated.

In embodiments that comprise a dosing arrangement and aerosol generators, at least one of the first and second the aerosol generators can be configured to vaporize or aerosolize at least one of the first and second component of the inhaling substance by heating or ultrasonics.

At least one of the dosing component(s) can be configured to dose at least a component of the inhaling substance or the inhaling substance in time intervals. The time intervals can be at most 100 ms, preferably at most 50 ms, more preferably at most 35 ms, preferably at most 25 ms, more preferably at most 20 ms, more preferably at most 15 ms, even more preferably at most 10 ms and most preferably at most 7 ms. The time intervals can be at least 1 ms, preferably at least 2 ms, preferably at least 3 ms, more preferably at least 4 ms, even more preferably at least 5 ms and even more preferably at least 6 ms. The time intervals can be between 1 ms and 15 ms, preferably between 2 ms and 20 ms, more preferably between 3 ms and 15 ms and most preferably between 5 ms and 10 ms. Thus, the dosing can be rather short and can supply the same amounts in pre-defined time intervals but not the entire time. This allows a sophisticated but rather simple dosing. By the time intervals of dosing, the time interval is understood during which a substance or a component is released.

The dosing arrangement can optionally be configured to heat at least a component of the inhaling substance or the inhaling substance to a temperature of more than 100° C., preferably at least 150° C., more preferably between 150° C. and 300° C., even more preferably between 180° C. and 260° C. and most preferably between 200° C. and 240° C.

In embodiments that comprising a first and second aerosol generator, the first and second reservoir and the respective first and second aerosol generators can be arranged in series along and in connection with the canal for delivering air. The connection can be at least indirect.

The system can further comprise at least one of a mouthpiece that is configured to be taken into the mouth by a user and a battery that is configured to deliver energy to the dosing arrangement. The mobile inhaler can further comprise at least one of a mouthpiece that is configured to be taken into the mouth by a user and a battery that is configured to deliver energy to the dosing arrangement.

The system can optionally further comprise a user interface with at least one of an activation switch, an LED, a display, a fingerprint sensor, a face recognition sensor, and a lip recognition sensor.

At least one reservoir of the plurality of reservoirs may preferably comprise at most 20 ml of a component of the inhaling substance, more preferably at most 10 ml of a component of the inhaling substance and still more preferably at most 5 ml of a component of the inhaling substance. Said reservoir may most preferably comprise at most 2 ml of a component of the inhaling substance. Said reservoir may preferably comprise the first component of the inhaling substance. Said first component may be nicotine.

At least one reservoir of the plurality of reservoirs may preferably comprise at most 20 ml of a component of the inhaling substance and at least 0.01 ml of the component of the inhaling substance, more preferably at most 10 ml and at least 0.1 ml of a component of the inhaling substance, still more preferably at most 5 ml and at least 1 ml of a component of the inhaling substance and most preferably at most 2 ml and at least 1.5 ml of a component of the inhaling substance, and wherein said reservoir is preferably comprising the first component of the inhaling substance and said component is preferably the first component of the inhaling substance.

The first component of the inhaling substance may comprise at least 2 mg nicotine per ml of liquid first component, preferably at least 5 mg/ml nicotine per liquid first component, more preferably at least 10 mg/ml nicotine per liquid first component, still more preferably at least 15 mg/ml nicotine per liquid first component and at the utmost preferably at least 18 mg/ml nicotine per liquid first component. The liquid first component refers to the first component of the inhaling substance in a liquid form, in particular to the first component under standard conditions.

The first component of the inhaling substance may comprise at most 100 mg/ml nicotine per liquid first component, preferably at most 80 mg/ml nicotine per liquid first component, still more preferably at most 60 mg/ml nicotine per liquid first component, still more preferably at most 40 mg/ml nicotine per liquid first component and utmost preferably at most 25 mg/ml nicotine per liquid first component.

Another aspect of the present invention is directed to a method for operating the system. The system is according to any of the preceding embodiments of the system. The method comprises delivering the inhaling substance by the system. The method comprises furthermore delivering at least the first component of the inhaling substance by the system according to a set of conditions.

In this disclosure, a set of conditions can comprise at least one or a plurality of conditions, wherein the set can furthermore comprise logical links of the results of the conditions, such as the logical conjunction, the logical disjunction or the negation of all or a part of the boolean results of some conditions or of the intermediate results obtained by the aforementioned conjunctions, disjunctions or negations.

The set of conditions can regulate at least one of a quantity and a general delivery of at least one of the first component and the inhaling substance. This option can be particularly helpful, considering the nicotine, nicotine derivate or other psychoactive substance in the first component of the inhaling substance. In particular, accidental delivery or delivery to users where e.g. an inhalation of a psychoactive substance is contraindicated could be avoided.

The method can furthermore comprise at least partially automatically regulating at least the amount the first component of the inhaling substance that is delivered over time. This regulating can be at least partially be performed by the system, however, the fully automatic regulation is preferred.

The amount of the first component of the inhaling substance can be controlled by a regulation model. The regulation model can be a (mathematical) model that is configured to be used to control the amount of the first component of the inhaling substance that is delivered. It can for example output the desired flow of the first component in mass unit per time unit to an element that then implements this output. The regulation model can, as another example, also output relative values, such as an increase or a decrease of the delivered amount of the first component of the inhaling substance.

The automatically regulating the amount of the first component that is delivered over time can be determining said amount of the first component by at least one of different approaches. The regulating of the amount of the first component that is delivered over time can be based on a pre-defined pattern or function, a pre-defined rule-set, and/or on a pattern based on data that is at least one of measured, generated and recorded by the system or by the mobile inhaler. It can also be based on a mathematical model or function based on data that is at least measured, generated and/or recorded by the system or the mobile inhaler, a machine learning or artificial intelligence model and/or further data. Those possible bases of the regulating of the amount of the first component are understood as detailed above in the context of the computing component of the system.

Said further data that can be a base or an approach for automatically regulating the amount of the first component that is delivered over time can comprise at least one or a plurality of the following data.

The further data can comprise indicators for a user's behaviour or habit and/or environmental factors such as people or devices around a user or a category or label associated with a user's environment, such as “place with smoking ban”, “station”, “hospital” or “place with vaping ban”, wherein this label can be suitable or configured to determine whether the delivery of the inhaling substance, the first or the second substance is legal.

The data can also comprise an external command from a user to raise, lower or adapt the amount of the first component in a composition of the inhaling substance or analogously an external command from a third party to raise, lower or adapt the amount of the first component in a composition of the inhaling substance. This third party can for example be medical personnel or a parent having the custody of an underage user.

The further data can also comprise an external command from a user to deliver, to deliver at all or to prevent delivering the inhaling substance and/or the first component of the inhaling substance.

The further data can furthermore comprise an external command from a third party to deliver, to deliver at all or to prevent delivering the inhaling substance and/or the first component of the inhaling substance.

The further data can also comprise data about a user's physiological state or at least a part thereof, such as a blood pressure, (an) indicator(s) for emotional stress, a medication, an intoxication, transpiration or diseases that the user has and/or a user's physiological reaction to a composition of the inhaling substance or at least a part of said reaction.

The further data that can be a base for the automatically regulating the amount of the first component can furthermore comprise a user's or the mobile inhaler's position, a time or date, an audio input, and/or data from wearable biological or medical sensors.

The regulating of the amount of the first component of the inhaling substance can be a reducing over a period of time.

The regulating can alternatively be an increasing over a period of time, for example in cases where a psychoactive drug is delivered for a palliative treatment of pain that is caused by a progressive disease.

The regulating of the amount of the first component of the inhaling substance can also be an adapting. When the regulating is an adapting, the system can for example adapt the amount of the delivered first component to the progress of a user who wants to reduce his/her consumption of the first component or a substance contained therein (such as nicotine), wherein an appropriate dosage might not always comprise a reduction of the first component of the inhaling substance. This could for example be the case when the user also reduces his/her use of inhalers in general. Another example would be a method to deliver a psychoactive substance to treat pain that requires treatment with varying doses of the psychoactive substance, e.g. because the severity of the pain or its cause varies.

The regulating of the amount of the first component of the inhaling substance can also be an adapting to a specified dose, such as a dose that was specified by medical personnel in the context of a patient's treatment, in particular when the user is the patient.

The regulating of the amount of the first component of the inhaling substance can furthermore also be an adapting to a condition of a user.

The method can comprise recording an inhalation of the inhaling substance.

The recording can comprise measuring the inhalation of the inhaling substance at least indirectly.

The recording can be performed during a period of time with at least one of a specified start and end.

The recording data can be started after a condition is met, such as an action of a person. For example, medical personnel, the user or a person bringing the system or the mobile inhaler into operation can perform an action that can start the recording.

The condition to meet in order to start said recording can be a single condition or a set of conditions linked by logical connections which then also forms a single condition. The condition can comprise a user input via at least one of a button, a finger print sensor, a tactile element and a microphone. The condition can also comprise an input via a device that is connected wirelessly or by direct physical contact, wherein said device is preferably an end user computer device, such as a personal computer, a smartphone, a PDA, a smart watch and a medical device. The condition can also comprise a first delivery of the inhaling substance, a first delivery of the first component of the inhaling substance and/or a passing of a certain time after a first delivery of the inhaling substance.

In this disclosure, an end user device is a computer device that is configured to be at least mostly used by one user at the same time. In the context of an end user computer device, an end user computer device can in particular be a device that comprises a computing component and that is configured to perform calculations or steps or sub-steps of computing methods.

At least indirectly measuring of the inhalation can comprise measuring at least one of acoustic waves that are emitted during the inhalation and/or measuring a pressure or a pressure difference during the inhalation. This measuring can also comprise detecting an activation signal of the user to deliver the inhaling substance or at least a component of the inhaling substance. The activation signal can for example be a pressing of a button, for example if the system is configured to deliver the inhaling substance or at least one of its components while this button is pressed, or if the button is configured to increase the delivery of the inhaling substance or of one of its components when pressed. The measuring can furthermore comprise at least one of measuring a flow of the inhaling substance and measuring a flow of a composition of air and the inhaling substance.

In some embodiments, recording the inhalation of the inhaling substance can comprise recording information.

This recording can comprise recording a delivered dose of the inhaling substance and or a composition of the inhaling substance.

This recording can also comprise recording at least one of a number of inhales during a use of the system, a frequency of inhales during a use of the mobile inhaler, a temporal length of at least one or a plurality of inhales and a pressure during an inhale, as well as another measure for a user's consuming behaviour during a use of the system.

This recording can comprise recording at least one of a pressure, a velocity and a (temporal) length of single deliveries of the inhaling substance or other information concerning single deliveries of the inhaling substance.

This recording can comprise recording at least one of dates of a delivery of the inhaling substance, a location of an inhale, such as a geographical location and other indicators for the circumstances of an inhalation.

The recording can also comprise at least partially and at least indirectly measuring the recorded features with the system or the mobile inhaler.

In some embodiments, the recording can also comprise using other devices, systems or components.

The method can furthermore comprise verifying a set conditions before delivering the first component of the inhaling substance. That can be for example verifying said set of conditions before activating the mobile inhaler, before activating the system or before activating elements that perform at least a portion of the step of delivering the first component of the inhaling substance.

Said verifying can be performed before every step of delivering the first component of the inhaling substance or only before some of the steps of delivering the first component of the inhaling substance.

The step of delivering the first component of the inhaling substance before which said set of conditions is verified can also alternatively be the first delivery of the first component of the inhaling substance. That is, said verifying the set of conditions before delivering the first component of the inhaling substance can be performed before delivering the first component of the inhaling substance for the first time.

Said verifying can also be performed only when further conditions are met, such as a certain period of time during which the mobile inhaler or the system was inactive, a certain period of time since the mobile inhaler or the system was switched off and/or used for the last time, a number of puffs or the delivered amount of at least one of the first and the second component of the inhaling substance. The latter two exemplary types of conditions would limit the effect of a single verifying of said set of conditions to a consumption, the former two exemplary types of conditions refer to temporal circumstances and correspond to automatically preventing a delivery of at least a portion of the inhaling substance after a certain period of time (if there is no further verifying).

Said set of conditions can comprise a condition concerning an age of a person, such as an age of a user of the mobile inhaler or of a user of the system. Such a condition can for example be that the user of the mobile inhaler is not underage.

Said set of conditions can comprise at least one condition referring to a presence of a document, such as an identity document, wherein said document preferably satisfies a further condition, such as providing a holder's age.

Said set can also comprise at least one condition referring to a person's identity or an indicator for the aforementioned, such as his/her fingerprint(s), a password, a personal code, a confirmation of a person's identity by a third party, a digital identity that is provided e.g. by a cryptographic key, a certificate or a state-issued digital proof of a person's identity, a person's voice or one or a set of biometric identifiers. The person to whose identity the condition refers does not need to be a user to whom the system delivers the inhaling substance, but the condition could also refer to a third person or group of persons, such as a person who may authorize a delivery of at least the first component to the inhaling user, such as medical personnel.

At least one condition of the set of conditions can also refer to an authenticity of a document, such as an identity document, in particular a document to which another condition of the set of conditions refers.

Said set of conditions can also comprise at least one condition referring to at least one of a presence of an end user computer device, such as a personal computer, a smartphone, a PDA or a smart watch.

Said set of conditions can also comprise at least one condition referring to a presence of an object that is configured for a at least one of unilateral, bilateral and multilateral wireless communication, such as an RFID card, a device configured for NFC communication, a device configured for radio communication, such as a device configured for Bluetooth™ communication, a mobile cellular phone, a wireless router, any other sender/receiver-combination that is configured for the exchange of wireless messages or a sender that at least broadcasts messages, and a presence of an object that is configured for communication via a physical contact, such as a dongle, a SIM-card, an SD-card, a chip card or a device configured for communication via USB.

Said set of conditions can also comprise at least one condition referring to a presence of a mean of payment, such as a credit card, a debit card or qualifying elements of the mean of payment, and a position or a set of positions, such as a position of the mobile inhaler, zones with smoking prohibition or allowance or an overlapping of a position of the mobile inhaler and said zones. In this context, the qualifying elements of a mean of payment are elements that are necessary for at least one of performing or authorizing a payment with the mean of payment, such as the card number, the expiration date and the card security code for some payment methods that work with credit cards, and for verifying the genuineness of a mean of payment, such as security features of banknotes.

Said set of conditions can also comprise at least one condition referring to a position or a set of positions, such as a position of the mobile inhaler, zones with smoking prohibition or allowance or an overlapping of a position of the mobile inhaler and said zones.

The set of conditions can comprise at least one condition that refers to at least one of a validity and a presence of a token and a DNA of the person.

The token can be a security token.

The token can be at least one of a software token and a hardware token.

The set of conditions can comprise at least one condition that refers to at least one of communication with an authentication device that is configured to be connected to the mobile inhaler or the system, an input device that is configured to transmit data to the system, an authorization by an authorizing entity, and communication with a server.

The authentication device can be connected to at least one of the system and the mobile inhaler.

The authentication device can be connected to the at least one of the system and the mobile inhaler via at least one of NFC, RFID, Bluetooth and another form of wireless communication.

The input device can be a device to input identification or verification data. The input device can be configured for verification of an ID. The input device can verify an ID.

The input device can connect the system and/or the mobile inhaler to another verification system, such as a reading device for an ID.

The communication with the server may comprise receiving data from the server. The server can generate the data based on data stored on the server.

The data stored on the data can comprise medical data, such as a health record of the patient and medical indications and/or contra-indications.

The data stored on the server can be demographical data of the user.

The data stored on the server can be legal data regarding an admissibility of using smoking devices at specified locations and/or times.

The method can comprise delivering the first component of the inhaling substance only if the verified set of conditions is matched.

The method can comprise delivering the first component of the inhaling substance only if the verified set of conditions is matched and preventing the step of delivering the first component of the inhaling substance otherwise.

The method can comprise verifying at least one condition of the set of conditions at least partially by an end user computer device that is connected to the mobile inhaler or to the system, such as a personal computer, a portable personal computer, a smart phone or a medical device comprising a computer unit.

The method can also comprise verifying at least one condition of the set of conditions at least partially by a server that is connected to the mobile inhaler or to the system, wherein the server is a device that is configured to perform computing steps for other devices that comprise a computing component or at least a communication component. A server can be a device such as a server computer, a cloud computing system or a server system.

The method can also comprise verifying at least one condition of the set of conditions at least partially by a server that is at least indirectly connected to the mobile inhaler or to the system via an end user computer device that is connected to the mobile inhaler.

The method can also comprise verifying at least one condition of the set of conditions at least partially by a computing component that the system comprises, such as the computing component described in the system embodiments that are discussed above.

The method can also comprise a combination of any of the preceding four verifying steps. That is for an example that all conditions that require only a low computing performance but ideally fast access to sensing devices attached to the mobile inhaler are verified by the system's computing component, whereas complex verifying steps such as verifying an identity document's security features are performed by a server.

In some embodiments, the method can comprise furthermore analysing at least parts of recorded data.

In such embodiments, the method can comprise deriving information on at least one of an inhaling behaviour and an inhaling habit.

The analysing in the method can furthermore comprise deriving at least one of a function depending on further parameters, a rule-set and a pattern for regulating the amount of the first component of the inhaling substance. This regulating refers to the automatically regulating the amount of the first component of the inhaling substance that is delivered over time, which was detailed above.

The method can furthermore comprise that the analysing is done when a certain amount of data, defined by a criterion such as at least one of a minimum number of inhales, a minimum time of recording or a minimum time of delivery, was recorded. This can for example increase a reliability of a result of the analysing.

In some embodiments, said regulating can be started after a condition is matched. This condition can be that a certain amount of data according to a criterion has been recorded, that an analysis result matches a certain criterion such as a reliability, or both.

In some embodiments, an indicator for verifying at least one condition of a set of conditions before delivering the first component of the inhaling substance or the result of this verifying can be displayed on at least one of a portion of the system, the mobile inhaler and a device linked to the system via a wireless or wired connection. The device linked to the system can be any device whose connection to the system or a portion thereof is discussed in the system embodiments in the context of displaying information. It can also be an end user computer device. The connection can be direct or indirect. A direct connection can be advantageous as it does not require a particular infrastructure, such as a WLAN-network or an internet connection. An indirect connection can be advantageous as it can easily display the information at a remote location, such as at a health care provider or at a device of an owner of the mobile inhaler or the system, wherein this device can for example be a mobile device such as a smartphone.

In some embodiments, data can be displayed on a device linked to the system via a wireless or wired connection. The connection can be direct. The direction can alternatively be indirect. Optional advantages of the preceding paragraph apply accordingly. A device linked to the system can be linked to any portion of the system, such as the mobile inhaler.

In such embodiments, the step of displaying data can comprise displaying at least a part of recorded data. That is, displaying data can for example comprise at least one data point of a plurality of recorded data points.

The step of displaying data can furthermore comprise at least one of pre-processing, analysing, filtering and agglomerating at least a part of recorded data.

The method can furthermore comprise displaying at least one of
the at least one result of the analysis of recorded data,
at least a part of the recorded data and
at least a part of the recorded data that is at least one of pre-processed, analysed, filtered and agglomerated.

In some embodiments, the method can comprise furthermore connecting the mobile inhaler or the system at least at some points in time to a software application that is installed on at least one device selected from an end user computer device, a personal computer, a router and a medical device. The method can comprise that the selected device introduces personal data of an individual user, such as at least one of age, gender, weight, location, working profiles, smoking habits and the user's emotional state. The method can comprise furthermore that the selected device makes this data available to at least one of a method that comprises the step of regulating the amount of the first component of the inhaling substance and a method that comprises verifying a set of conditions. The method that comprises verifying the set of conditions can be in particular any of the abovementioned methods that comprise verifying a set of conditions before delivering at least the first component of the inhaling substance. The method that comprises the step of regulating the amount of the first component of the inhaling substance can be any of the abovementioned methods for at least partially automatically regulating at least the amount of the first component of the inhaling substance that is delivered over time. At least one step of the method regulating at least the amount of the first component of the inhaling substance that is delivered over time can at least partially be performed by at least one of the system, the mobile inhaler, the selected device, a server and a server to which the system is connected via the selected device. This explanation is to be understood in analogy to the explanation concerning performing at least steps of the verifying of a set of at least one or a plurality of conditions on different devices. The emotional state, which can be introduces by the selected device, can be deduced for example from audio input, analysing the voice profile of the user, or from an analysis of the user's use of an end user device, such as his/her tone in messages sent to other people via the end user device.

The personal data of the individual user can comprise at least one of the age, the gender, the weight, the location, the working profiles, the smoking habits and the user's emotional state.

The personal data of the individual user introduced by the selected device can also comprise at least one of demographic data, smoking history data of the user, data relating to a smoking behaviour of the user which are sensed by the mobile inhaler, and empirical data.

Empirical data can for example be data from scientific studies and/or clinical trials.

The selected device can comprise a data storage comprising at least parts of the introduced data. The selected device can also forward at least parts of the introduced data. The selected device can optionally also retrieve at least parts of data that it introduces from a third device or system, such as a server, a network attached storage, a cloud system or a data storage device, wherein that third device or that third system is connected to the selected device at least at some points in time.

In some embodiments, the system can be associated with a software application that is installed on an end user computer device that is configured to at least one of update, configure and modify the system or a portion thereof, such as at least a part of a software on the mobile inhaler. The system is considered to be associated with a device or a software application, if it is connected to this device or software application at least at some points in time. This connection can comprise exchanging data between the device/software application and the mobile inhaler, and the data can be data that has a further function apart from the data necessary to establish, maintain or terminate the connection. This connection can be direct, such as via USB or an end user device that also acts as WLAN-router, or indirect, such as a connection in a WLAN with a supplementary router. The latter can be advantageous in terms of a simple setup, if the network via which the indirect connection is to be established is already available. The former can be advantageous in case that such a network is not available or that it comprises restrictions which require further steps to establish a connection or to exchange data between the system and the end user computer device.

In some embodiments, the method can comprise supplementary steps. A supplementary step is to transfer data that was recorded or at least parts of this data from the system to a computer device and/or a computer system. Another step is to perform a calculation that is at least a part of the step of automatically regulating the amount of the first component of the inhaling substance that is delivered over time according to any of the preceding embodiments on the respective computer device and/or computer system. A further supplementary step is to transfer at least a part of a result or a plurality of results of said calculation back to the system.

In some embodiments, the method can preferably comprise using the system comprising at least one delivering restriction and at least one restriction element. In such embodiments, the method can comprise the step of delivering at least one of the first component and the second component of the inhaling substance only from liquid container(s) that comprise(s) at least one restriction element, and/or if there is another authorization. This other authorization can be the same other authorization that is detailed above.

The delivering restrictions and restriction elements that are used in the method can each independently from each other be according to at least one of the disclosed options for delivering restrictions and/or restriction elements described above, such as the delivering restrictions that are specific to a subset of the plurality of reservoirs, the delivering restriction and the respective restriction element that comprise at least the shape of at least one liquid container, the delivering restriction and the respective restriction element that comprise at least one connector that is configured to connect at least one fluid container to the mobile inhaler and/or the delivering restriction and the respective restriction element that comprise at least an electronic element configured for at least an identification the respective liquid container. The system that is used can comprise the mobile inhaler with at least one delivering restriction. The system that is used can comprise liquid container(s) that comprise at least one restriction element.

The method can furthermore comprise the step of delivering at least one of the first component and the second component of the inhaling substance only if at least a one of the delivering restriction(s) of the mobile inhaler and at least one of the restriction element(s) of at least one liquid container match and/or if there is another authorization.

For restriction elements that comprise an electronic element configured at least for an identification of the respective liquid container, matching means that at least a property of the information provided by the electronic element, such as its identity, a cryptographically signed identity or a cryptographically signed information about the content(s) of the reservoir(s) in the liquid container, is communicated to the mobile inhaler or another portion of the system and matches a condition. This condition does not necessarily need to be verified by the mobile inhaler or the other portion of the system, it can also be verified by a computer device that is at least indirectly connected to the system, such as a server that stores information about distributed liquid containers.

The method can optionally also comprise the step of delivering at least one of the first component and the second component of the inhaling substance only if for each of the at least one or the plurality of liquid containers, at least one of the system's delivering restriction(s) and at least one of the restriction element(s) of the respective liquid container match and/or if there is another authorization.

The method can optionally also comprise the step of delivering at least one of the first component and the second component of the inhaling substance only if all delivering restriction(s) are each matched by at least one of the restriction element(s) of the at least one or the plurality of liquid containers respectively and/or if there is another authorization.

It is underlined that in the above-mentioned methods comprising matching of at least one delivering restriction and at least one restriction element, as well as in the systems configured to perform such methods, the other authorization for delivery is an optional feature of the method or the system. That is, an optional embodiment of the present invention is also to deliver at least the first component of the inhaling substance only if the conditions in the different methods regarding the matching of the restriction element(s) and the delivering restriction(s) in the respectively detailed ways are met.

The method may comprise furthermore counting at least one number of inhales since a point in time and storing the at least one number of inhales. The at least one number of inhales can be stored on at least one of the system, a portion thereof, such as an electronic element that one of the at least one or the plurality of liquid containers comprises, and a third device, such as a smart handheld device, a mobile computer or a dongle, wherein this third device is configured for wired and/or wireless communication. The third device can be directly or indirectly connected to the system. The latter can optionally be advantageous in case that the third device is a server or a computer device that is not in proximity of the mobile inhaler.

One of the at least one number of inhales since a point in time can be a number of inhales of the inhaling substance comprising a specific component since a point in time. This number of inhales can for example be a number of inhales of the inhaling substance that comprises the first component since a point in time.

One of the at least one number of inhales since a point in time can also be a number of inhales of the inhaling substance comprising a component from a specific reservoir or from a specific liquid container since a point in time, such as a number of inhales of the inhaling substance comprising a component from a reservoir that comprises the first component of the inhaling substance since a point in time. In another example, this number of inhales can also be a number of inhales since a point in time that comprise a component from a liquid container with a certain identification as detailed in the context of the restriction elements.

One of the at least one number of inhales since a point in time can also be a number of inhales since a first activation of the system or a portion thereof, such as the mobile inhaler or one of the at least one or the plurality of liquid containers.

The method can also comprise storing at least two of the number of inhales since a first activation of the system or a portion thereof, the number of inhales of the inhaling substance comprising a component from a specific reservoir or from a specific liquid container since a point in time, and the number of inhales of the inhaling substance comprising a specific component since a point in time

The method can comprise verifying a set of conditions before delivering the first component of the inhaling substance as detailed above. The method can furthermore comprise storing an unlocking state, wherein the unlocking state is a result of the step of verifying, such as “true”, “false”, “unlocked” or “not unlocked”, or an indicator for this result.

The unlocking state can be stored on a third device.

The unlocking state can furthermore be communicated from the third device to system or a portion thereof, such as the mobile inhaler or at least one of the at least one or the plurality of liquid containers.

The third device can be at least one of a device configured for wireless communication, such as a mobile handheld device, a mobile computer or an RFID-card and a device configured for wired communication such as a dongle, chipcard, an integrated circuit with an appropriate packaging or an SD-card.

The third device can also be an end user computer device.

The unlocking state or parts thereof can also be stored on plurality of devices. As an example, the unlocking state can be stored in parts on different devices, e.g. by storing two values on two devices, wherein these values indicate that the system is unlocked if the values are equal or satisfy a condition such as “the first number is three times the second number”.

The method can also comprise storing the unlocking state on at least one or a plurality of elements that the system or a portion thereof, such as the mobile inhaler or at least one of the at least one or the plurality of liquid containers, comprises.

The unlocking state can be stored at least on a data storage element that one of the at least one or the plurality of liquid containers comprises.

The unlocking state can also be stored at least on a data storage element that the mobile inhaler comprises.

That is, the unlocking state can also be stored at least on both elements, an element that a liquid container comprises or on an element that the mobile inhaler comprises.

The unlocking state can also be stored at least on a data storage element that another portion of the system comprises.

The unlocking state can be stored using a cryptographic method, such as signing the data. The data can at least relate to or indicate the unlocking state.

Furthermore, storing the unlocking state can at least be performed by storing data. The data can indicate the unlocking state. That is, the unlocking state can also be stored indirectly, such as by storing the availability of functions that are unlocked.

One of the element or the elements on which the unlocking state or data indicating the unlocking state are stored can furthermore be at least one of an electronic storage element, an electrical or electronical component that is damaged and a mechanical element that is deformed, removed or added. The electronic storage element can be an element such as a memory chip, a microcontroller, a microprocessor or at least a portion thereof. The electrical or electronical component that is damaged can be an element such as a fuse that is burnt or a microcontroller or circuit that is short-circuited. This damage can be to an extent that significantly impacts a function of the electrical or electronical component or completely destroys the component, so that the damage can be clearly detected. The mechanical element that is deformed, removed or added can be an element such as a piece of metal that is bent by mechanical, thermal or magnetic forces or a piece of metal that links two electric contacts and that is removed or added. The element used for storing the unlocking state can be configured for storing the unlocking state or data relating to it.

Storing the unlocking state can also comprise at least one of enabling an enabling element that enables at least a part of delivering the first component of the inhaling substance and disabling a disabling element that inhibits at least a part of delivering the first component of the inhaling substance. That can be, if all enabling elements that are used for at least a part of delivering the first component of the inhaling substance are enabled and all elements that inhibit at least a part of delivering the first component of the inhaling substance, the system can be at least partially unlocked. The unlocking may nevertheless still require another method step. If at least one of the enabling elements is not enabled or at least one of the disabling elements is not disabled, the system may accordingly be not unlocked. The enabling and disabling of the respective elements can optionally be performed according to the set conditions that is verified before delivering the first component of the inhaling substance.

In the following, optional and non-exclusive embodiments of the enabling element are discussed.

The enabling element can be an electrical contact, such as a contact between a switch and a controller accepting an input signal from the switch to trigger delivering the inhaling substance or the first component of the inhaling substance.

The enabling element can also be a mechanical connection configured to conduct a gas, a liquid or a mixture of those two. This mechanical connection can be a connection such as a connection that is configured to conduct air, the inhaling substance, a component of the inhaling substance or a fluid that is used to generate the inhaling substance or a component thereof.

The enabling element can also be a thermally conductive connection, such as a connection between a heating element and a vaporizing element configured to conduct heat to the vaporizing element. For example, the vaporizing element can be configured to vaporize at least the first component of the inhaling substance.

The enabling element can also be a configuration, a software or a portion of any of those two, that is at least necessary to deliver at least the first component of the inhaling substance. This can be for example a configuration of a sensor, such as a sensor to detect an inhaling of the user, a software for a (micro-)controller, a configuration of the regulation model controlling the amount of the first component of the inhaling substance that is delivered over time, or a portion of any of those configurations or this software. The configuration, the software or the portion of any of those two can be enabled by providing a data element that is configured to be used in a cryptographic process, such as a cryptographic key, a cryptographically signed element or a cryptographic certificate.

The enabling element can also be a data element that is configured to be used in cryptographic process, such as a cryptographic key, a cryptographically signed element or a cryptographic certificate.

In the following, optional and non-exclusive embodiments of the disabling element are discussed.

The disabling element can be an electrical contact preventing the system from delivering the first component of the inhaling substance. This electrical contact can be for example a contact such as an electrical contact that prevents a micro-controller from booting or a contact that shunts a component or a sensor that is necessary for delivering the first component of the inhaling substance.

The disabling element can also be a mechanical connection or a mechanical obstruction, such as a connection configured to bypass elements that are necessary to deliver at least the first component of the inhaling substance or a valve in a connection configured to conduct the inhaling substance or a component thereof in liquid, partially liquid or gaseous form.

The disabling element can also be an element that obscures or inhibits a part of or all communication between portions of the system, such as a communication between portions of the mobile inhaler or a communication between the mobile inhaler and one of the at least one or the plurality of liquid containers. This element can for example be a resistor, a capacitor, an inductor, an inverter or an electronic filter, such as an LC circuit. This element can be for example integrated into a portion of a transmission element configured for the communication that is inhibited.

The disabling element can also be an element that inhibits a sensing or counting of inhales, if the sensing or counting of the inhales is required for at least the delivery of the first component of the inhaling substance. For an example, this disabling element can be an element that prevents the device configured to sense a measure related to the step of delivering at least one of a component of the inhaling substance and the inhaling substance from said sensing.

In some embodiments, delivering the inhaling substance can comprise the step of providing the first component of the inhaling substance during inhaling and the step of providing at least the second component of the inhaling substance during inhaling. At least the first component can be dosed in intervals of at most 50 ms during inhaling.

In some embodiments, the first component can be automatically regulated over time at least by regulating the number of inhales comprising the first component. Regulating the number of inhales comprising the first component can comprise regulating the number of inhales during which the first component is dosed.

The amount of the first component can be regulated by delivering the first component in time intervals during one inhale and by reducing the number of time intervals for a single inhale over time.

In some embodiments, the mobile inhaler can be a battery driven mobile device.

In some embodiments, the mobile inhaler can be an electronic cigarette.

In some embodiments, at least the second component of the inhaling substance can be a liquid to be vaporized to provide an aerosol as a carrier component together with air. The carrier component can be a carrier component for the first component of the inhaling substance.

In some embodiments, the method can further comprise delivering air from a canal to a mouthpiece. At least one of the first and second component of the inhaling substance can be delivered as aerosols to the canal.

The first and the second component of the inhaling substance can both be delivered as aerosols. They can furthermore be delivered in series to the canal.

At least one of the aerosols from the first and the second component of the inhaling substance can be directly delivered into the canal.

In some embodiments, said components of the inhaling substance, that is at least the first and the second component, can be liquids. In such embodiments, the method can comprise furthermore vaporizing the first liquid and/or the second liquid.

The vaporized liquid(s) can be vaporized by at least one of heating and ultrasonic.

In some embodiments, the method can comprise releasing at least the first component of the inhaling substance or the inhaling substance in time intervals. In such embodiments, the length of each time interval can be at most 100 ms, preferably at most 50 ms, more preferably at most 35 ms, preferably at most 25 ms, more preferably at most 20 ms, more preferably at most 15 ms, even more preferably at most 10 ms and most preferably at most 7 ms.

In some embodiments, at least the first component of the inhaling substance or the inhaling substance can be released in time intervals of at least 1 ms, preferably at least 2 ms, preferably at least 3 ms, more preferably at least 4 ms, even more preferably at least 5 ms, even more preferably at least 6 ms.

In some embodiments, at least the first component of the inhaling substance or the inhaling substance can be released in time intervals. In such embodiments, the time interval can be between 1 ms and 15 ms, preferably between 2 ms and 20 ms, more preferably between 3 ms and 15 ms and most preferably between 5 ms and 10 ms.

In some embodiments, at least one of the first component and the second component of the inhaling substance can be heated to a temperature of more than 100° C., preferably at least 150° C., more preferably between 150° C. and 300° C., even more preferably between 180° C. and 260° C. and most preferably between 200° C. and 240° C.

Another aspect of the invention is a therapeutic method with the step of applying any of the aforementioned methods or using any of the aforementioned systems. The therapeutic method can be used for but is not limited to the support of smokers trying to quit. The therapeutic method can also be directed to the delivery of psychoactive substances for an adapted treatment of pain.

An optional embodiment of the invention is a system that can be configured to carry out any of the aforementioned methods.

The system can be according to any of the systems that are described above, and these systems can furthermore be configured to carry out any of the aforementioned methods.

Another optional aspect of the invention is directed to a computer program product comprising instructions, which, when the program is executed by a mobile inhaler, can cause the mobile inhaler to perform the method steps, which have to be executed on the mobile inhaler according to any method described above. In such embodiments, the mobile inhaler can be according to any of the embodiments of a mobile inhaler described above, wherein the mobile inhaler can be compatible to said method embodiment.

A further optional aspect of the invention can comprise a computer program product comprising instructions, which, when the program is executed by an end user computer device, can cause the end user computer device to perform the method steps according to any method embodiment, which have to be executed on the end user computer device. In such embodiments, the end user computer device can be according to any system embodiment comprising an end user computer device that is compatible to said method embodiment. In particular, the end user device can be computer device that is configured to be at least mostly used by one user at a time.

### NUMBERED EMBODIMENTS

Below, system embodiments will be discussed. These embodiments abbreviated by the letter “S” followed by a number. Whenever reference is herein made to “system embodiments”, these embodiments are meant.
S1 A system comprising
   a mobile inhaler (1) that is configured to be simultaneously connected to at least one or a plurality of liquid containers (17), and to deliver an inhaling substance (10), wherein the inhaling substance (10) comprises at least one of an amount of a first component (11) and an amount of a second component (12).
S2 The system according to the preceding embodiment,
   wherein the mobile inhaler (1) further comprises
   at least one or a plurality of connector(s) (80) configured for connection of the mobile inhaler (1) to the at least one or the plurality of liquid containers (17) and for intaking of at least a portion of each of the liquid container(s)'s (17) content(s).
S3 The system according to the preceding embodiment,
   wherein the system comprises furthermore
   the at least one or the plurality of liquid containers (17), wherein the at least one or the plurality of liquid containers (17) comprises a plurality of reservoirs, wherein each reservoir comprises a component (11, 12) of the inhaling substance (10).
S4 The system according to any of the preceding embodiments that comprise at least one reservoir,
   wherein the reservoirs each comprise mutually different components (11, 12) of the inhaling substance.
S5 The system according to the preceding embodiment,
   wherein the concentration of at least one chemical substance in each component (11, 12) differs at least by a factor of ten for each pair of components (11, 12), wherein this differing substance can be different for each pair of components (11, 12).
S6 The system according to any of the preceding embodiments,
   wherein the components (11, 12) of the inhaling substance (10) that are stored in the liquid container(s) (17) are at least partially liquid.
S7 The system according to any of the preceding embodiments,
   wherein each liquid container (17) comprises at least one or a plurality of connector(s) (80) configured for connection of the respective liquid container (17) to the mobile inhaler (1).
S8 The system according to any of the preceding embodiments,
   wherein the at least one of the at least one or the plurality of liquid containers (17) is at least one liquid container (17) that comprises a plurality of reservoirs.
S9 The system according to any of the preceding embodiments but the last,
   wherein the at least one or the plurality of liquid containers (17) is a plurality of liquid containers (17) that comprises a plurality of reservoirs.
S10 The system according to any of the preceding embodiments,
   wherein the system comprises a dosing component (50) configured to perform at least the dosing of at least a component (11, 12) of the inhaling substance (10).
S11 The system according to the preceding embodiment,
   wherein the system comprises a dosing component (50) configured to perform the dosing of at least one of the first component (11) and the second component (12) of the inhaling substance (10).
S12 The system according to the preceding embodiment,
   wherein the dosing component (50) is configured to perform the dosing of at least the first component (11) and the second component (12) of the inhaling substance (10) independently from each other.
S13 The system according to the preceding embodiment,
   wherein the dosing component (50) comprises two dosing mechanisms that perform the dosing of at least the first component (11) and at least the second component (12) respectively.
S14 The system according to any of the preceding system embodiments,
   wherein the system furthermore comprises a computing component (2).
S15 The system according to the preceding system embodiment,
   wherein the mobile inhaler (1) comprises at least a portion of the computing component (2).
S16 The system according to any of the preceding system embodiments that comprise the computing component (2),
   wherein the computing component (2) is at least configured to perform calculations that are necessary for at least one of a dosing and a delivery of at least one of the inhaling substance (10), the first component (11) and the second component (12) of the inhaling substance (10).
S17 The system according to any of the preceding system embodiments that comprise the computing component (2),
   wherein the computing component (2) is an integrated circuit.
S18 The system according to any of the preceding system embodiments that comprise the computing component (2),
   wherein the computing component (2) is at least one of a micro-controller, a microprocessor, an ASIC, an FPGA or a CPLD.
S19 The system according to any of the preceding system embodiments,
   wherein at least one of the mobile inhaler (1) and at least one of the liquid container(s) (17) comprises furthermore a data storage component (3) that is at least configured to store data.
S20 The system according to the preceding system embodiment,
   wherein data storage component (3) is at least one selected from a list of
   (a) an electronic storage element, such as a memory chip, a microcontroller, a microprocessor or at least a portion thereof,
   (b) an integrated circuit,
   (c) a flash memory component,
   (d) a RAM component,
   (e) a read-only memory component,
   (f) a hard disk,
   (g) an electrical or electronical component that is destroyed, such as a fuse that is burnt or a microcontroller or an electronical circuit that is short-circuited, and
   (h) a mechanical element that is deformed, removed or added, such as a metal that is bent by mechanical, thermal or magnetic forces or a piece of metal that links two electric contacts is removed or added.
S21 The system according to any of the preceding system embodiments that comprise the computing component (2),
   wherein the computing component (2) is at least configured to perform at least a part of a calculation for regulating the amount of at least the first component (11) that is delivered over time based on at least one of
   (a) a pre-defined pattern or function,
   (b) a pre-defined rule-set,
   (c) a pattern based on data that is at least one of measured, generated and recorded by the mobile inhaler (1),
   (d) a mathematical model or function based on data that is at least measured, generated and/or recorded by the mobile inhaler (1),
   (e) a machine learning or artificial intelligence model; and
   (f) further data.
S22 The system according to any of the preceding system embodiments,
   wherein the system comprises at least one or a plurality of information input elements selected from
   (a) a localisation component,
   (b) a sound sensor, such as a microphone,
   (c) an interaction element for a user or a third party configured to signal to the mobile inhaler (1) to deliver or to deliver at all the inhaling substance (10) or a component thereof (11, 12), or to raise, lower and/or adapt at least the delivered amount of the inhaling substance (10) or of a component thereof (11, 12), such as a physical knob or button or a button on a touch-sensitive display,
   (d) at least one sensing device to sense the user's physiological state,
   (e) a clock,
   (f) a timer,
   (g) a wearable biological or medical sensor,
   (h) a database comprising at least labels for positions,
   (i) a device to configured to sense a measure related to the step of delivering at least one of a component (11, 12) of the inhaling substance (10) and the inhaling substance (10), such as a pressure sensing device, a sound sensing device, an airflow sensing device or a signal from the user to perform the delivery,
   (j) a finger print sensor, and
   (k) a tactile sensor.
S23 The system according to the preceding embodiment,
   wherein the mobile inhaler (1) is configured for connection to at least one, a plurality or all of the at least one or a plurality of information input elements.
S24 The system according to the penultimate embodiment,
   wherein the mobile inhaler (1) comprises at least one, a plurality or all of the at least one or a plurality of information input elements.
S25 The system according to any of the preceding system embodiments,
   wherein system comprises furthermore an interface (4) suitable to connect the system to at least one of a computer device, an integrated circuit and a data storage device, by at least one of a wired and a wireless connection.
S26 The system according to the preceding system embodiment,
   wherein the interface (4) is furthermore configured to connect the system to at least one of a computer device, an integrated circuit and a data storage device.
S27 The system according to the preceding system embodiment,
   wherein the interface (4) is an interface for wired communication, such as a USB-interface or an interface configured to physically connect the mobile inhaler (1) to at least one of a dongle, a SIM-card, an SD-card, a chip card, a magnetic stripe and any other integrated circuit element.
S28 The system according to the penultimate system embodiment,
   wherein the interface (4) is an interface for wireless communication, such as an interface for radio communication, such as interfaces to communicate with RFID-chips or communication systems marketed as Bluetooth™ or Wi-Fi™, NFC communication, communication via mobile internet or via cellular network services.
S29 The system according to any of the four preceding system embodiments,
   wherein the interface (4) is an interface configured for direct or indirect connection.
S30 The system according to any of the preceding system embodiments,
   wherein the system comprises a document checking component (5) configured to verify and/or check a property provided by at least one of an official identity document, a mean of payment and another document to confirm the holder's identity.
S31 The system according to the preceding system embodiment,
   wherein the document checking component (5) is a computing device with an appropriate sensing device and an appropriate software, wherein the computing device is connected to the mobile inhaler (1) at least indirectly.
S32 The system according to the preceding system embodiment,
   wherein the computing device connected to the mobile inhaler (1) is a mobile device, such as a portable personal computer or a smartphone, furthermore comprising an appropriate application or set of applications.
S33 The system according to the preceding system embodiment,
   wherein the application or at least one application of the set of applications on the mobile device is configured to communicate data to a remote server in order to verify and/or check a property provided by at least one of an identity document and at least one mean of payment.
S34 The system according to any of the preceding system embodiments,
   wherein the mobile inhaler (1) comprises furthermore a connection configured to transfer data to/from an input interface element (6),
   wherein the input interface element (6) is configured to receive data input from a user, such as input of a PIN, a password or voice.
S35 The system according to the preceding embodiment,
   wherein the system comprises furthermore the input interface element (6) that is configured to receive data input from a user.
S36 The system according to any of the two preceding embodiments,
   wherein the mobile inhaler (1) comprises furthermore the input interface element (6) that is configured to receive data input from a user.
S37 The system according to any of the three preceding embodiments,
   wherein the input interface element (6) is at least one of an interface of a mobile device and an interface of a computer device,
   wherein the respective device is connected to the mobile inhaler (1) at least at one point in time.
S38 The system according to any of the preceding system embodiments,
   wherein the mobile inhaler (1) comprises a connection component to an output interface component (7).
S39 The system according to any of the preceding embodiments that comprise the connection component to an output interface component (7),
   wherein the system comprises furthermore the output interface component (7).
S40 The system according to any of the preceding embodiments that comprise the connection component to an output interface component (7),
   wherein the output interface component (7) is configured to display information relating to the mobile inhaler (1), a delivery of the inhaling substance (10) and/or an inhalation, information relating to a recording and/or a duration of an inhalation, a summary or an analysis thereof or information relating to at least one liquid container (17) that is, was or can be connected to the mobile inhaler (1).
S41 The system according to any of the preceding embodiments that comprise the connection component to an output interface component (7),
   wherein the output interface component (7) is at least one of
   (a) a visual interface device, such as an LED, an LED-array, a screen or a projector,
   (b) an acoustic output device, such as a speaker, a buzzer or another device configured to play audio data, and
   (c) a haptic element, such as a vibrating element.
S42 The system according to any of the preceding embodiments that comprise the connection component to an output interface component (7),
   wherein the output interface component (7) is at least one of
   a computer device and a display device that is connected to a mobile inhaler (1), such as a smartphone, a personal computer, a printer, a screen or a virtual reality headset.
S43 The system according to any of the preceding system embodiments,
   wherein the mobile inhaler (1) comprises furthermore a connection component that is configured to connect the mobile inhaler (1) at least to a controlling control at least indirectly and at least at one point in time, preferably at least at some points in time.
S44 The system according to the preceding system embodiment,
   wherein the system comprises furthermore the controlling control that is at least indirectly and at least at some points in time connected to the mobile inhaler (1).
S45 The system according to any of the two preceding system embodiments,
   wherein the controlling control is configured to at least influence the delivery of at least the first component (11) of the inhaling substance (10).
S46 The system according to any of the three preceding system embodiments,
   wherein the controlling control is implemented in software; and
   wherein the controlling control is furthermore at least partially running on at least one of
   (a) a smart handheld device,
   (b) a remote server,
   (c) a cloud computing system,
   (d) a medical device, and
   (e) another computer or computer system carrying out the task of running the controlling control.
S47 The system according to any of the preceding system embodiments that comprise at least one connector (80),
   wherein the mobile inhaler (1) comprises furthermore at least one or a plurality of delivering restriction(s) (61), at least one of the liquid container(s) (17) comprise(s) at least one or a plurality of matching restriction element(s) (60), and each of the at least one delivering restriction(s) (61) is a feature configured to prevent delivering at least one component (11, 12) of the inhaling substance (10) by the mobile inhaler (1) from a liquid container (17) without the respective restriction element(s) (60).
S48 The system according to any of the preceding system embodiments that comprise at least one of a restriction element (60) and a delivering restriction (61),
   wherein the mobile inhaler (1) is configured to deliver at least one component (11, 12) of an inhaling substance (10) only if the at least one or the plurality of delivering restriction(s) (61) are matched by at least a part of the restriction element(s) (60) of at least one of the respective liquid container(s) (17), and/or if there is another authorization.
S49 The system according to any of the preceding system embodiments that comprise at least one of a restriction element (60), at least one delivering restriction (61) and a plurality of reservoirs in at least one liquid container (17), wherein the mobile inhaler (1) is configured to deliver at least one component (11, 12) of the inhaling substance (10) only if the delivering restriction(s) (61) are matched by at least a part of the restriction element(s) (60) of at least one of the respective liquid container(s) (17), wherein furthermore at least one of the delivering restriction(s) (61) is specific to a to a subset of the plurality of reservoirs and the liquid container(s) (17) comprising this subset, and/or if there is another authorization.
S50 The system according to any of the preceding system embodiments that comprise at least one of a restriction element (60) and a delivering restriction (61),
   wherein the mobile inhaler (1) is configured to deliver at least one component (11, 12) of the inhaling substance (10) only if the at least one or the plurality of delivering restriction(s) (61) are matched by at least a part of the at least one or the plurality of restriction element(s) (60) of all liquid container(s) (17) that are connected to the mobile inhaler (1), and/or if there is another authorization.
S51 The system according to any of the preceding system embodiments that comprise at least one of a restriction element (60) and a delivering restriction (61),
   wherein at least one of the at least one restriction element(s) (60) comprises the shape of at least one liquid container (17).
S52 The system according to any of the preceding system embodiments that comprise at least one of a restriction element (60) and a delivering restriction (61),
   wherein at least one of the at least one of the mobile inhaler's (1) delivering restriction(s) (61) comprises an element that limits the liquid container(s) (17) that can be connected to the mobile inhaler (1) at least based on their shape.
S53 The system according to the preceding embodiment,
   wherein at least one of the mobile inhaler's (1) delivering restriction(s) (61) comprises an element that limits the liquid container(s) (17) that can be connected to the mobile inhaler (1) at least based on their shape,
   furthermore, this element comprises a cavity in the mobile inhaler (1) that is configured to accommodate at least one of the liquid container(s) (17) with one or a plurality of restriction element (60), such as a cavity with a limited width and/or a limited length or a cavity with a particular geometry of at least a portion of the cavity, such as a geometry of at least a portion of the cavity selected from a cone-shape, a paraboloid of revolution, a hyperboloid of revolution, a spheroid, a pyramidal shape, a cuboid shape and a freeform surface; and/or the geometry of the cavity comprising furthermore a bar, a screw thread, a bolt, a hole or an asymmetry.
S54 The system according to any of the preceding system embodiments that comprise at least one of a restriction element (60) and a delivering restriction (61),
   wherein at least one of the at least one restriction element(s) (60) comprise(s) the connector of at least one of the fluid container(s) (17) to at least one of the mobile inhaler's (1) connector(s) (80).
S55 The system according to any of the preceding system embodiments that comprise at least one of a restriction element (60) and a delivering restriction (61),
   wherein at least one of the mobile inhaler's (1) delivering restriction(s) (61) comprises at least one of the mobile inhaler's connector(s) (80).
S56 The system according to the preceding embodiment,
   wherein at least one of the mobile inhaler's (1) delivering restriction(s) (61) that comprise(s) at least one of the mobile inhaler's connector(s) (80) comprises furthermore the shape of the at least one of the mobile inhaler(s) (1) connector(s) (80), such as a polygon shape, a round shape, a round shape with an irregularity such as an indentation or a splined shaft shape.
S57 The system according to any of the two preceding embodiments,
   wherein at least one of the mobile inhaler's (1) delivering restriction(s) (61) that comprise(s) at least one of the mobile inhaler's connector(s) (80) comprises furthermore a connector (80) configured to require a minimum pressure in at least one of the reservoir(s) in at least one of the liquid container(s) (17), such as an overpressure.
S58 The system according to any of the preceding system embodiments that comprise at least one of a restriction element (60) and a delivering restriction (61),
   wherein at least one restriction element (60) of at least one of the liquid container(s) (17) comprises an electronic element configured at least for an identification of the respective liquid container (17) by at least one of wired communication, such as a USB-interface or an interface configured to physically connect the mobile inhaler (1) to at least one of a dongle, a SIM-card, an SD-card, a chip card, a magnetic stripe and any other integrated circuit element, and wireless communication, such as an interface for radio communication, such as interfaces to communicate with RFID-chips or communication systems marketed as Bluetooth™ or Wi-Fi™, or NFC communication.
S59 The system according to any of the preceding system embodiments that comprise at least one of a restriction element (60) and a delivering restriction (61),
   wherein at least one of the mobile inhaler's (1) delivering restriction(s) (61) comprise(s) an element configured to identify at least one liquid container (17) by an electronic element that is mounted to the liquid container (17) and that is configured at least for an identification of the liquid container (17) by at least one of wired and wireless communication according to the preceding embodiment.
S60 The system according to any of the preceding system embodiments,
   wherein the system comprises at least two reservoirs in at least one liquid container (17), each reservoir comprising one component (11, 12) of the inhaling substance (10); and a dosing arrangement (51) that is configured to automatically regulate the amount of the first component (11) that is delivered over time according to a set of conditions (20) and that comprises a dosing component (50) according to any of the preceding embodiments that comprise a dosing component (50).
S61 The system according to the preceding system embodiment,
   wherein the dosing arrangement (51) is configured to dose at least the first component (11) in time intervals of at most 200 ms.
S62 The system according to any of the two preceding system embodiments,
   wherein the dosing arrangement (51) is configured to regulate the amount of the first component (11) that is delivered over time based on at least one of
   (a) a pre-defined pattern or function,
   (b) a pre-defined rule-set,
   (c) a pattern based on data that is at least one of measured, generated and recorded by the mobile inhaler (1),
   (d) a mathematical model or function based on data that is at least measured, generated and/or recorded by the mobile inhaler (1),
   (e) a machine learning or artificial intelligence model; and
   (f) further data.
S63 The system according to any of the preceding system embodiments,
   wherein the mobile inhaler (1) is preferably a battery driven mobile device and more preferably an electronic cigarette or e-cigarette.
S64 The system according to any of the preceding system embodiments that comprise the dosing arrangement (51),
   wherein the dosing arrangement (51) comprises a first aerosol generator that is configured to vaporize the first component (11) of the inhaling substance (10) to a first aerosol, preferably so as to provide at least air as a carrier component together with the first aerosol; and a second aerosol generator that is configured to vaporize the second component (12) of an inhaling substance (10) to a second aerosol, preferably so as to provide at least air as a carrier component together with the second aerosol.
S65 The system according to any of the preceding system embodiments that comprise the dosing arrangement (51),
   wherein the mobile inhaler (1) further comprises a mouth piece (54) and a canal (55) that is configured to supply air to the mouth piece (54), and wherein at least one of the first and second aerosol generator(s) are configured to deliver at least one of the first and second aerosols to the canal (55).
S66 The system according to any of the preceding system embodiments that comprise the dosing arrangement (51),
   wherein the dosing arrangement (51) comprises furthermore a controller that is configured to control at least one of the first and second aerosol generators.
S67 The system according to the preceding system embodiment,
   wherein the controller is configured to at least control at least one of the activation(s) of the first and second aerosol generators and an amount of aerosol generated.
S68 The system according to any of the preceding system embodiments that comprise the dosing arrangement (51) and the aerosol generators,
   wherein at least one of the first and second the aerosol generators are configured to vaporize or aerosolize at least one of the first and second component (11, 12) of the inhaling substance (10) by heating or ultrasonics.
S69 The system according to any of the preceding system embodiments that comprise the dosing component (50),
   wherein the dosing component (50) is configured to dose at least a component (11, 12) of the inhaling substance (10) or the inhaling substance (10) in time intervals of at most 100 ms, preferably at most 50 ms, more preferably at most 35 ms, preferably at most 25 ms, more preferably at most 20 ms, more preferably at most 15 ms, even more preferably at most 10 ms and most preferably at most 7 ms.
S70 The system according to any of the preceding system embodiments that comprise the dosing component,
   wherein the dosing component is configured to dose at least a component (11, 12) of the inhaling substance (10) or the inhaling substance (10) in time intervals of at least 1 ms, preferably at least 2 ms, preferably at least 3 ms, more preferably at least 4 ms, even more preferably at least 5 ms, even more preferably at least 6 ms.
S71 The system according to any of the preceding system embodiments that comprise the dosing component,
   wherein the dosing component is configured to dose at least a component (11, 12) of the inhaling substance (10) or the inhaling substance (10) in time intervals of between 1 ms and 15 ms, preferably between 2 ms and 20 ms, more preferably between 3 ms and 15 ms and most preferably between 5 ms and 10 ms.
S72 The system according to any of the preceding system embodiments that comprise the dosing arrangement (51),
   wherein the dosing arrangement (51) is configured to heat at least a component (11, 12) of the inhaling substance (10) or the inhaling substance (10) to a temperature of more than 100° C., preferably at least 150° C., more preferably between 150° C. and 300° C., even more preferably between 180° C. and 260° C. and most preferably between 200° C. and 240° C.
S73 The system according to any of the preceding system embodiments that comprise the first and second aerosol generator,
   wherein the first and second reservoir and the respective first and second aerosol generators are arranged in series along and in connection with a canal (55) for delivering air.
S74 The system according to any of the preceding system embodiments that comprise the dosing arrangement (51),
   further comprising at least one of a mouthpiece (54) that is configured to be taken into the mouth of a user and a battery that is configured to deliver energy to the dosing arrangement (51).
S75 The system according to any of the preceding system embodiments,
   further comprising a user interface with at least one of an activation switch, an LED, a display, a fingerprint sensor, a face recognition sensor, a lip recognition sensor.
S76 The system according to any of the preceding system embodiments,
   wherein at least one reservoir of the plurality of reservoirs preferably comprises at most 20 ml of a component (11, 12) of the inhaling substance (10), more preferably at most 10 ml of a component (11, 12) of the inhaling substance (10), still more preferably at most 5 ml of a component (11, 12) of the inhaling substance (10) and most preferably at most 2 ml of a component (11, 12) of the inhaling substance (10), and wherein said reservoir is preferably comprising the first component (11) of the inhaling substance.
S77 The system according to the preceding system embodiment,
   wherein at least one reservoir of the plurality of reservoirs preferably comprises at most 20 ml of a component (11, 12) of the inhaling substance (10) and at least 0.01 ml of the component (11, 12) of the inhaling substance (10), more preferably at most 10 ml and at least 0.1 ml of a component (11, 12) of the inhaling substance (10), still more preferably at most 5 ml and at least 1 ml of a component (11, 12) of the inhaling substance (10) and most preferably at most 2 ml and at least 1.5 ml of a component (11, 12) of the inhaling substance (10), and wherein said reservoir is preferably comprising the first component (11) of the inhaling substance.
S78 The system according to any of the preceding system embodiments,
   wherein the first component (11) of the inhaling substance (10) comprises at least 2 mg nicotine per ml of liquid first component (11), preferably at least 5 mg/ml nicotine per liquid first component (11), more preferably at least 10 mg/ml nicotine per liquid first component (11), still more preferably at least 15 mg/ml nicotine per liquid first component (11) and at the utmost preferably at least 18 mg/ml nicotine per liquid first component (11).
S79 The system according to any of the preceding system embodiments,
   wherein the first component (11) of the inhaling substance (10) comprises at most 100 mg/ml nicotine per liquid first component (11), preferably at most 80 mg/ml nicotine per liquid first component (11), still more preferably at most 60 mg/ml nicotine per liquid first component (11), still more preferably at most 40 mg/ml nicotine per liquid first component (11) and utmost preferably at most 25 mg/ml nicotine per liquid first component (11).

Below, method embodiments will be discussed. These embodiments are abbreviated by the letter “M” followed by a number. Whenever reference is herein made to “method embodiments”, these embodiments are meant.
M1 A method particularly for operating the system according to any of the preceding system embodiments (1), comprising
   delivering the inhaling substance (10) by the system,
   wherein the system delivers (1) at least the first component (11) of the inhaling substance (10) according to a set of conditions (20).
M2 The method according to the preceding method embodiment,
   wherein said set of conditions (20) regulates at least one of a quantity and a general delivery of at least one of the first component (11) and the inhaling substance (10).
M3 The method according to any of the preceding method embodiments,
   comprising at least partially automatically regulating at least the amount the first component (11) of the inhaling substance (10) that is delivered over time.
M3 The method according to the preceding method embodiment,
   wherein the amount of the first component (11) is controlled by a regulation model.
M4 The method according to any of the two preceding method embodiments,
   wherein automatically regulating the amount of the first component (11) that is delivered over time follows at least one of
   (a) a pre-defined pattern or function,
   (b) a pre-defined rule-set,
   (c) a pattern based on data that is at least one of measured, generated and recorded by the system or the mobile inhaler (1),
   (d) a mathematical model or function based on data that is at least measured, generated and/or recorded by the system or the mobile inhaler (1),
   (e) a machine learning or artificial intelligence model; and
   (f) further data.
M5 The method according to the preceding method embodiment,
   wherein said further data comprises at least one or a plurality of
   (a) indicators for a user's behaviour or habit,
   (b) environmental factors such as people or devices around a user or a category or label associated with a user's environment,
   (c1) an external command from a user to raise, lower or adapt the amount of the first component (11) in a composition of the inhaling substance (10),
   (c2) an external command from a third party to raise, lower or adapt the amount of the first component (11) in a composition of the inhaling substance (10),
   (c3) an external command from a user to deliver, to deliver at all or to prevent delivering the inhaling substance (10) and/or the first component (11) of the inhaling substance (10),
   (c4) an external command from a third party to deliver, to deliver at all or to prevent delivering the inhaling substance (10) and/or the first component (11) of the inhaling substance (10),
   (d) a user's physiological state or at least a part thereof, such as a blood pressure, (an) indicator(s) for emotional stress, medication, intoxication, transpiration or diseases,
   (e) a user's physiological reaction to a composition of the inhaling substance (10) or at least a part of said reaction,
   (f) a user's or the mobile inhaler's (1) position,
   (g) a time or date,
   (h) audio input, and
   (i) data from wearable biological or medical sensors.
M6 The method according to any of the preceding method embodiments that comprise at least partially automatically regulating at least the amount the first component (11) that is delivered over time,
   wherein said regulating is a reducing over a period of time.
M7 The method according to any of the preceding method embodiments that comprise at least partially automatically regulating at least the amount the first component (11) that is delivered over time,
   wherein said regulating is an increasing over a period of time.
M8 The method according to any of the preceding method embodiments that comprise at least partially automatically regulating at least the amount the first component (11) that is delivered over time,
   wherein said regulating is an adapting.
M9 The method according to any of the preceding method embodiments that comprise at least partially automatically regulating at least the amount the first component (11) that is delivered over time,
   wherein said regulating is an adapting to a specified dose.
M10 The method according to any of the preceding method embodiments that comprise at least partially automatically regulating at least the amount the first component (11) that is delivered over time,
   wherein said regulating is an adapting to a condition of a user.
M11 The method according to any of the preceding method embodiments,
   comprising furthermore recording an inhalation of the inhaling substance (10).
M12 The method according to the preceding method embodiment,
   wherein recording the inhalation of the inhaling substance (10) comprises measuring the inhalation of the inhaling substance (10) at least indirectly.
M13 The method according to any of the two preceding method embodiments,
   wherein said recording is performed during a period of time with at least one of a specified start and end.
M14 The method according to any of the preceding method embodiments that comprise recording data,
   wherein said recording data is started after a condition is met, such as an action of a person.
M15 The method according to the preceding embodiment,
   wherein said condition to meet in order to start said recording is at least one of
   (a) a user input via at least one of a button, a finger print sensor, a tactile element and a microphone,
   (b) an input via a device that is connected wirelessly or by direct physical contact, wherein said device is preferably an end user computer device, such as a personal computer, a smartphone, a PDA, a smart watch and a medical device,
   (c) a first delivery of the inhaling substance (10),
   (d) a passing of a certain time after a first delivery of the inhaling substance (10),
   (e) a first delivery of the first component (11) of the inhaling substance (10), and
   (f) a combination thereof.
M16 The method according to any of the preceding method embodiments that comprise measuring the inhalation of the inhaling substance (10) at least indirectly,
   wherein at least indirectly measuring the inhalation comprises measuring at least one of
   (a) a flow of the inhaling substance (10),
   (b) a flow of a composition of air and the inhaling substance (10),
   (c) acoustic waves that are emitted during the inhalation,
   (d) a pressure difference during the inhalation, and
   (e) an activation signal from a user, such as a pressing of a button.
M17 The method according to any of the preceding method embodiments that comprise recording the inhalation of the inhaling substance (10),
   wherein said recording comprises recording at least one of
   (a) a delivered dose of the inhaling substance (10),
   (b) dates of a delivery of the inhaling substance (10),
   (c) a composition of the inhaling substance (10),
   (d) at least one of a pressure, a velocity and a (temporal) length of single deliveries of the inhaling substance (10),
   (e) a number of inhales during a use of the system,
   (f) a frequency of inhales during a use of system,
   (g) a temporal length of at least one or a plurality of inhales,
   (h) a pressure during an inhale,
   (i) a location of an inhale, such as a geographical location, and
   (j) any combination of those aforementioned features.
M18 The method according to the preceding embodiment,
   wherein said recording comprises at least partially and at least indirectly measuring the recorded features with the system or the mobile inhaler (1).
M19 The method according to any of the preceding method embodiments,
   comprising furthermore verifying a set of conditions before delivering the first component (11) of the inhaling substance (10).
M20 The method according to the preceding embodiment,
   wherein said verifying is performed before every step of delivering of the first component (11) of the inhaling substance (10) or only before some of the steps of delivering of the first component (11) of the inhaling substance (10).
M21 The method according to the penultimate method embodiment,
   wherein the step of delivering of the first component (11) of the inhaling substance (10) before which said set of conditions is verified is the first delivery of the first component (11) of the inhaling substance (10).
M22 The method according to any of the preceding method embodiments that comprise the features of M19,
   wherein said verifying is performed only when further conditions are met, such as a certain period of time during which the mobile inhaler (1) or the system was inactive, a certain period of time since the mobile inhaler (1) or the system was switched off and/or used for the last time, a number of puffs or the delivered amount of at least one of the first and the second component (11, 12) of the inhaling substance (10).
M23 The method according to any of the preceding method embodiments that comprise the features of M19,
   wherein said set of conditions comprises a condition concerning an age of a person.
M24 The method according to any of the preceding method embodiments that comprise the features of M19,
   wherein the set of condition comprises at least one condition that refers to at least one of
   (a) a presence of a document, such as an identity document, wherein said document preferably satisfies a further condition, such as providing a holder's age,
   (b) a person's identity or an indicator for the aforementioned, such as his fingerprint(s), a password, a personal code, a confirmation of a person's identity by a third party, a digital identity that is provided e.g. by a cryptographic key, a certificate or a state-issued digital proof of a person's identity, a person's voice or one or a set of biometric identifiers,
   (c) an authenticity of a document, such as an identity document,
   (d) a presence of an end user computer device, such as a personal computer, a smartphone, a PDA or a smart watch,
   (e) a presence of an object that is configured for a at least one of unilateral, bilateral and multilateral wireless communication, such as an RFID card, a device configured for NFC communication, a device configured for radio communication, such as a device configured for Bluetooth™ communication, a mobile cellular phone, a wireless router, any other sender/receiver-combination that is configured for the exchange of wireless messages or a sender that at least broadcasts messages,
   (f) a presence of an object that is configured for communication via a physical contact, such as a dongle, a SIM-card, an SD-card, a chip card or a device configured for communication via USB,
   (g) a presence of a mean of payment, such as a credit card, a debit card or the qualifying elements thereof, and
   (h) a position or a set of positions, such as a position of the mobile inhaler (1), zones with smoking prohibition or allowance or an overlapping of a position of the mobile inhaler (1) and said zones.
M25 The method according to any of the preceding method embodiments with the features of M19,
   wherein the set of conditions comprises at least one condition that refers to at least one of
   (a) a validity and a presence of a token, and
   (b) a DNA of the person.
M26 The method according to any of the preceding method embodiments with the features of M19,
   wherein the set of condition comprises at least one condition that refers to at least one of
   (a) communication with an authentication device that is configured to be connected to the mobile inhaler or the system,
   (b) an input device that is configured to transmit data to the system,
   (c) an authorization by an authorizing entity, and
   (d) a communication with a server.
M27 The method according to any of the preceding method embodiments that comprise the features of M19,
   comprising furthermore delivering the first component (11) of the inhaling substance (10) only if the verified set of conditions is matched.
M28 The method according to any of the preceding method embodiments that comprise the features of M19,
   comprising furthermore delivering the first component (11) of the inhaling substance (10) only if the verified set of conditions is matched and preventing the step of delivering the first component (11) of the inhaling substance (10) otherwise.
M29 The method according to any of the preceding method embodiments that comprise the features of M19,
   comprising verifying at least one condition of the set of conditions at least partially by an end user computer device that is connected to the mobile inhaler (1) or to the system, such as a personal computer, a portable personal computer, a smart phone or a medical device comprising a computer unit.
M30 The method according to any of the preceding method embodiments that comprise the features of M19,
   comprising verifying at least one condition of the set of conditions at least partially by a server that is connected to the mobile inhaler (1) or to the system, such as a server computer, a cloud computing system or a server system.
M31 The method according to any of the preceding method embodiments that comprise the features of M19,
   comprising verifying at least one condition of the set of conditions at least partially by a server that is at least indirectly connected to the mobile inhaler (1) or to the system via an end user computer device that is connected to the mobile inhaler (1).
M32 The method according to any of the preceding method embodiments that comprise the features of M19,
   comprising verifying at least one condition of the set of conditions at least partially by a computing component that the system comprises, such as the computing component (2) according to the system embodiment 14 and its depending embodiments.
M33 The method according to any of the preceding method embodiments that comprise the features of M19,
   comprising combining any of the verifying steps according to the preceding four method embodiments.
M34 The method according to any of the preceding method embodiments that comprise recording or measuring data on an inhaling of the inhaling substance (10) by the mobile inhaler (1) or the system,
   comprising furthermore analysing at least parts of recorded data.
M35 The method according to the preceding embodiment,
   comprising furthermore
   deriving information on at least one of an inhaling behaviour and an inhaling habit.
M36 The method according to any of the preceding method embodiments that comprise analysing at least parts of recorded data,
   wherein said analysing comprises
   deriving at least one of a function depending on further parameters, a rule-set and a pattern for regulating the amount of the first component (11) of the inhaling substance (10).
M37 The method according to any of the preceding method embodiments that comprise recording data and analysing at least parts of recorded data,
   wherein said analysing is performed when a certain amount of data, defined by a criterion such as at least one of a minimum number of inhales, a minimum time of recording or a minimum time of delivery, was recorded.
M38 The method according to any of the preceding method embodiments that comprise recording data, analysing at least parts of recorded data and regulating at least the amount of the first component (11) of the inhaling substance (10),
   wherein said regulating is only started after a at least one of
   (a) a certain amount of data according to a criterion has been recorded, and
   (b) an analysis result matches a certain criterion such as a reliability.
M39 The method according to any of the preceding method embodiments that comprise verifying a set of conditions before delivering the first component (11) of the inhaling substance (10),
   comprising furthermore displaying at least an indicator for said verifying or its result on at least one of a portion of the system, the mobile inhaler (1) and a device linked to the system via a wireless or wired connection.
M40 The method according to any of the preceding method embodiments that comprise recording data,
   comprising furthermore displaying data on a device linked to the system via a wireless or wired connection, wherein the connection is direct or indirect.
M41 The method according to the preceding embodiment,
   wherein the step of displaying data comprises displaying at least a part of the recorded data.
M42 The method according to the preceding embodiment,
   wherein the step of displaying data comprises at least one of pre-processing, analysing, filtering and agglomerating at least a part of the recorded data.
M43 The method according to any of the preceding method embodiments that comprise analysing and recording data,
   comprising furthermore displaying at least one of the at least one result of the analysis, at least a part of the recorded data and at least a part of the recorded data that is at least one of pre-processed, analysed, filtered and agglomerated.
M44 The method according to any of the preceding method embodiments,
   wherein the method comprises furthermore
   connecting the mobile inhaler (1) or the system at least at some points in time to a software application that is installed on at least one device selected from an end user computer device, a personal computer, a router and a medical device, introducing personal data of an individual user by the selected device and the selected device making this data available to at least one of
   a method that comprises the step of regulating the amount of the first component (11) of the inhaling substance (10) and a method that comprises verifying a set of conditions, such as the methods according to embodiment M19 and its dependent embodiments.
M45 The method according to the preceding method embodiment, wherein the personal data of the individual user introduced by the selected device comprise at least one of age, gender, weight, location, working profiles, smoking habits and the user's emotional state.
M46 The method according to any of the two preceding method embodiments, wherein the personal data of the individual user introduced by the selected device comprise at least one of
   (a) demographic data,
   (b) smoking history data of the user,
   (c) data relating to a smoking behaviour of the user which are sensed by the mobile inhaler, and
   (d) empirical data.
M47 The method according to any of the preceding method embodiments with the features of M44,
   wherein the selected device is at least one of
   (a) comprising a data storage comprising at least parts of the introduced data, (b) forwarding at least parts of the introduced data, and
   (c) retrieving at least parts of the introduced data from a third device or a third system, such as a server, a network attached storage, a cloud system or a data storage device,
   wherein that third device or that third system is connected to the selected device.
M48 The method according to any of the preceding method embodiments,
   wherein the system is associated with a software application that is installed on an end user computer device that is configured to at least one of update, configure and modify the system or a portion thereof, such as at least a part of a software on the mobile inhaler (1).
M49 The method according to any of the preceding method embodiments that comprise recording data and at least partially automatically regulating the amount of the first component (11) of the inhaling substance (10) that is delivered over time,
   comprising furthermore transferring the recorded data or at least a part thereof from the system to a computer device and/or a computer system, performing a calculation that is at least a part of the automatically regulating the amount of the first component (11) of the inhaling substance (10) that is delivered over time according to any of the preceding embodiments and transferring at least a part of (a) result(s) of said calculation back to the system.
M50 The method according to any of the preceding method embodiments,
   comprising furthermore preferably using the system according to any of the system embodiments that comprise at least one delivering restriction (61) and at least one restriction element (60), and delivering at least one of the first component (11) and the second component (12) of the inhaling substance (10) only from liquid container(s) (17) that comprise(s) at least one restriction element (60), and/or if there is another authorization.
M51 The method according to the preceding method embodiment,
   wherein the delivering restriction(s) (61) and the restriction elements (60) are according to the system embodiment S47 or any of its depending embodiments.
M52 The method according to any of the preceding method embodiments that comprise the features of M50,
   comprising furthermore delivering at least one of the first component (11) and the second component (12) of the inhaling substance (10) only if at least a one of the delivering restriction(s) (61) and at least one of the restriction element(s) (60) of at least one liquid container (17) match and/or if there is another authorization.
M53 The method according to any of the preceding method embodiments that comprise the features of M50,
   comprising furthermore delivering at least one of the first component (11) and the second component (12) of the inhaling substance (10) only if for each of the at least one or the plurality of liquid containers (17), at least one of the system's delivering restriction(s) (61) and at least one of the restriction element(s) (60) of the respective liquid container (17) match and/or if there is another authorization.
M54 The method according to any of the preceding method embodiments that comprise the features of M50,
   comprising furthermore delivering at least one of the first component (11) and the second component (12) of the inhaling substance (10) only if all delivering restriction(s) (61) are each matched by at least one of the restriction element(s) (60) of the at least one or the plurality of liquid containers (17) respectively and/or if there is another authorization.
M55 The method according to any of the preceding method embodiments,
   comprising furthermore counting at least one number of inhales since a point in time and storing the at least one the number of inhales on at least one of the system, a portion thereof, such as an electronic element that one of the at least one or the plurality of liquid containers (17) comprises, and a third device, such as a smart handheld device, a mobile computer or a dongle, wherein this third device is configured for wired and/or wireless communication.
M56 The method according to the preceding method embodiment,
   wherein one of the at least one number of inhales since a point in time is a number of inhales of the inhaling substance (10) comprising a specific component (11, 12) since a point in time, such as a number of inhales of the inhaling substance (10) that comprises the first component (11) since a point in time.
M57 The method according to any of the two preceding method embodiments,
   wherein one of the at least one number of inhales since a point in time is a number of inhales of the inhaling substance (10) comprising a component (11, 12) from a specific reservoir or from a specific liquid container (17) since a point in time, such as a number of inhales of the inhaling substance (10) comprising a component from a reservoir that comprises the first component (11) of the inhaling substance (10) since a point in time.
M58 The method according to any of the three preceding method embodiments,
   wherein the number of inhales since a point in time is at least the number of inhales since a first activation of the system or a portion thereof, such as the mobile inhaler (1) or one of the at least one or the plurality of liquid containers (17).
M59 The method according to any of the four preceding embodiments,
   comprising storing at least two of
   (a) the number of inhales since a first activation of the system or a portion thereof,
   (b) the number of inhales of the inhaling substance (10) comprising a component (11, 12) from a specific reservoir or from a specific liquid container (17) since a point in time, and
   (c) the number of inhales of the inhaling substance (10) comprising a specific component (11, 12) since a point in time.
M60 The method according to any of the preceding method embodiments that comprise the features of M19,
   comprising storing an unlocking state (40),
   wherein the unlocking state (40) is a result of the step of verifying the set of conditions or an indicator for this result.
M61 The method according to the preceding method embodiment,
   wherein the unlocking state (40) is stored on a third device.
M62 The method according to the preceding embodiment,
   wherein the unlocking state (40) is furthermore communicated from the third device to the system or a portion thereof.
M63 The method according to the preceding method embodiment,
   wherein the third device is at least one of a device configured for wireless communication, such as a mobile handheld device, a mobile computer or an RFID-card and a device configured for wired communication such as a dongle, chipcard, an integrated circuit with an appropriate packaging or an SD-card.
M64 The method according to the penultimate method embodiment,
   wherein the third device is an end user computer device.
M65 The method according to any of the preceding method embodiments that comprise the features of M60,
   wherein the unlocking state (40) or parts thereof are stored on a plurality of devices,
   wherein at least one or all of the plurality of devices are preferably according to any of the third devices in the preceding two method embodiments.
M66 The method according to the any of the three preceding method embodiments,
   comprising storing the unlocking state (40) on at least one or a plurality of element(s) that the system or a portion thereof, such as the mobile inhaler (1) or at least one of the at least one or the plurality of liquid containers (17), comprises.
M67 The method according to the preceding method embodiment,
   wherein the unlocking state (40) is stored at least on a data storage element that one of the at least one or the plurality of liquid containers (17) comprises.
M68 The method according to any of the two preceding method embodiments,
   wherein the unlocking state (40) is stored at least on a data storage element that the mobile inhaler (1) comprises.
M69 The method according to any of the two preceding embodiments,
   wherein the unlocking state (40) is stored using a cryptographic method, such as signing data.
M70 The method according to any of the three preceding embodiments,
   wherein storing the unlocking state (40) is at least performed by storing data that indicates the unlocking state (40).
M71 The method according to the preceding embodiment,
   wherein one of the element(s) on which the unlocking state or data indicating the unlocking state are stored is at least one of
   (a) an electronic storage element, such as a memory chip, a microcontroller, a microprocessor or at least a portion thereof,
   (b) an electrical or electronical component that is damaged, such as a fuse that is burnt or a microcontroller or circuit that is short-circuited, and
   (c) a mechanical element that is deformed, removed or added, such as a piece of metal that is bent by mechanical, thermal or magnetic forces or a piece of metal that links two electric contacts and that is removed or added.
M72 The method according to any of the preceding method embodiments with the features of M65,
   wherein storing the unlocking state (40) comprises at least one of
   (a) enabling an enabling element that enables at least a part of delivering the first component (11) of the inhaling substance (10), and
   (b) disabling a disabling element that inhibits at least a part of delivering the first component (11) of the inhaling substance (10).
M73 The method according to the preceding method embodiment,
   wherein the enabling element is at least one of
   (a) an electrical contact,
   (b) a mechanical connection configured to conduct a gas, a liquid or a mixture of those two,
   (c) a thermally conductive connection,
   (d) a configuration, a software or a part of any of those two, that is at least necessary to deliver at least the first component (11) of the inhaling substance (10), wherein the configuration, the software or the part of any of those two can be enabled by providing a data element that is configured to be used in a cryptographic process, and
   (e) a data element that is configured to be used in a cryptographic process.
M74 The method according to any of the preceding two method embodiments,
   wherein the disabling element is at least one of
   (a) an electrical contact preventing the system from delivering the first component of the inhaling substance,
   (b) mechanical connection or a mechanical obstruction,
   (c) an element that obscures or inhibits a part of or all communication between portions of the system,
   (d) an element that inhibits a sensing or counting of inhales, if this sensing or counting is required for at least the delivery of the first component (11) of the inhaling substance (10).
M75 The method according to any of the preceding method embodiments,
   wherein delivering the inhaling substance (10) is comprising the steps of
   (a) providing the first component (11) of the inhaling substance (10) during inhaling; and
   (b) providing at least the second component (12) of the inhaling substance (10) during inhaling;
   wherein at least the first component (11) is dosed in intervals of at most 50 ms during inhaling.
M76 The method according to any of the preceding method embodiments that comprise the features of M3,
   wherein the first component (11) is automatically regulated over time at least by regulating the number of inhales comprising the first component (11).
M77 The method according to any of the preceding two method embodiments,
   wherein the amount of the first component (11) is regulated by delivering the first component (11) in time intervals during one inhale and by reducing the number of time intervals for a single inhale over time.
M78 The method according to any of the preceding method embodiments wherein the mobile inhaler (1) is a battery driven mobile device.
M79 The method according to any of the preceding method embodiments wherein the mobile inhaler (1) is an electronic cigarette.
M80 The method according to any of the preceding method embodiments,
   wherein at least the second component (12) of the inhaling substance (10) is a liquid to be vaporized to provide an aerosol as a carrier component together with air.
M81 The method according to any of the preceding method embodiments,
   wherein the method further comprises delivering air from a canal (55) to a mouthpiece (54) and wherein at least one of the first (11) and second component of the inhaling substance (10) are delivered as aerosols to the canal (55).
M82 The method according to the preceding method embodiment,
   wherein the first (11) and the second component (12) of the inhaling substance (10) are both delivered as aerosols and wherein they are delivered in series to the canal (55).
M83 The method according to the preceding method embodiment,
   wherein at least one of the aerosols from the first (11) and the second component (12) of the inhaling substance are directly delivered into the canal (55).
M84 The method according to any of the preceding method embodiments,
   wherein said components (11, 12) of the inhaling substance (10) are liquids, and
   wherein the method comprises furthermore vaporizing the first liquid and/or the second liquid.
M85 The method according to the preceding method embodiment,
   wherein at least one or all of the vaporized liquid(s) are vaporized by at least one of heating and ultrasonic.
M86 The method according to any of the preceding method embodiments,
   wherein the method comprises furthermore releasing at least the first component (11) of the inhaling substance (10) or the inhaling substance (10) in time intervals,
   wherein the length of each time interval is at most 100 ms, preferably at most 50 ms, more preferably at most 35 ms, preferably at most 25 ms, more preferably at most 20 ms, more preferably at most 15 ms, even more preferably at most 10 ms and most preferably at most 7 ms.
M87 The method according to any of the preceding method embodiments, wherein the method comprises furthermore releasing at least the first component (11) of the inhaling substance (10) or the inhaling substance (10) in time intervals,
   wherein the time interval is at least 1 ms, preferably at least 2 ms, preferably at least 3 ms, more preferably at least 4 ms, even more preferably at least 5 ms, even more preferably at least 6 ms.
M88 The method according to the any of the preceding method embodiments,
   wherein the method comprises furthermore releasing at least the first component (11) of the inhaling substance (10) or the inhaling substance (10) in time intervals,
   wherein the time interval is between 1 ms and 15 ms, preferably between 2 ms and 20 ms, more preferably between 3 ms and 15 ms and most preferably between 5 ms and 10 ms.
M89 The method according to any of the preceding method embodiments,
   wherein at least one of the first component (11) and the second (12) component of the inhaling substance (10) is/are heated to a temperature of more than 100° C., preferably at least 150° C., more preferably between 150° C. and 300° C., even more preferably between 180° C. and 260° C. and most preferably between 200° C. and 240° C.
M90 A therapeutic method with the step of applying any of the preceding system embodiments or method embodiments.

Below, further system embodiments will be discussed. Those system embodiments continue the list of system embodiments started above.
S80 A system that is configured to carry out any of the methods described in the method embodiments.
S81 The system according to the preceding system embodiment, that is furthermore according to any of the preceding system embodiments.

Below, computer program product embodiments will be discussed. These embodiments are abbreviated by the letter “P” followed by a number. Whenever reference is herein made to “program embodiments”, these embodiments are meant.
P1 A computer program product comprising instructions, which, when the program is executed by a mobile inhaler (1), cause the mobile inhaler (1) to perform the method steps according to any of the method embodiments, which have to be executed on the mobile inhaler (1),
   wherein the mobile inhaler (1) is according to any of the system embodiments that comprise a mobile inhaler (1) that is compatible to said method embodiment.
P2 A computer program product comprising instructions, which, when the program is executed by an end user computer device, cause the end user computer device to perform the method steps according to any of the method embodiments, which have to be executed on the end user computer device.

It is to be noted that other embodiments with further different arrangements of structural components are covered by the present invention.

Reference numbers and letters appearing between parentheses in the claims, identifying features described in the embodiments and illustrated in the accompanying drawings, are provided as an aid to the reader as an exemplification of the matter claimed. The inclusion of such reference numbers and letters is not to be interpreted as placing any limitations on the scope of the claims.

The term “at least one of a first option and a second option” is intended to mean the first option or the second option or the first option and the second option.

Whenever a relative term, such as “about”, “substantially” or “approximately” is used in this specification, such a term should also be construed to also include the exact term. That is, e.g., “substantially straight” should be construed to also include “(exactly) straight”.

Whenever steps were recited in the above or also in the appended claims, it should be noted that the order in which the steps are recited in this text may be the preferred order, but it may not be mandatory to carry out the steps in the recited order. That is, unless otherwise specified or unless clear to the skilled person, the orders in which steps are recited may not be mandatory. That is, when the present document states, e.g., that a method comprises steps (A) and (B), this does not necessarily mean that step (A) precedes step (B), but it is also possible that step (A) is performed (at least partly) simultaneously with step (B) or that step (B) precedes step (A). Furthermore, when a step (X) is said to precede another step (Z), this does not imply that there is no step between steps (X) and (Z). That is, step (X) preceding step (Z) encompasses the situation that step (X) is performed directly before step (Z), but also the situation that (X) is performed before one or more steps (Y1), . . . , followed by step (Z). Corresponding considerations apply when terms like “after” or “before” are used.

### FIGURE DESCRIPTION

### Brief Description of the Figures

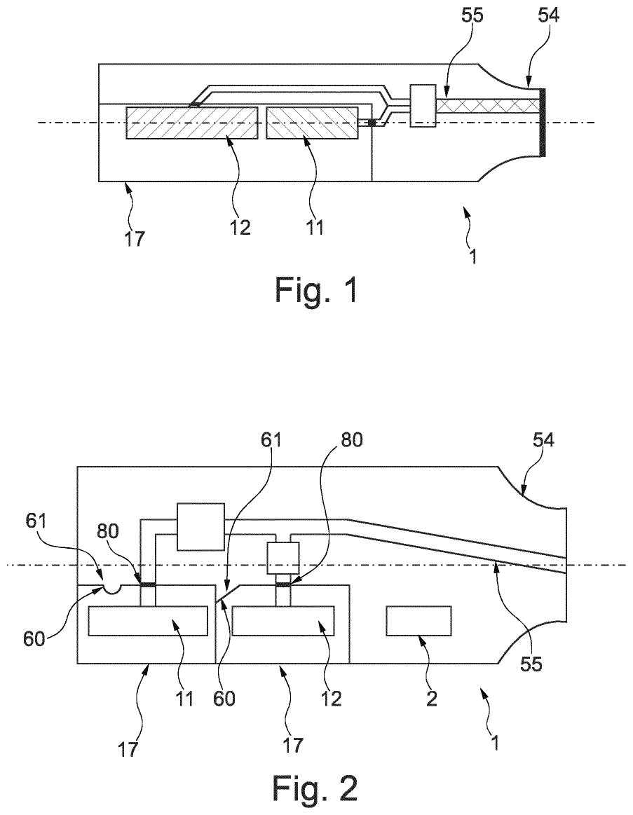

FIG. 1 the mobile inhaler with a liquid container that comprises two components of the inhaling substance

FIG. 2 the mobile inhaler with two liquid containers that comprise each one component of the inhaling substance

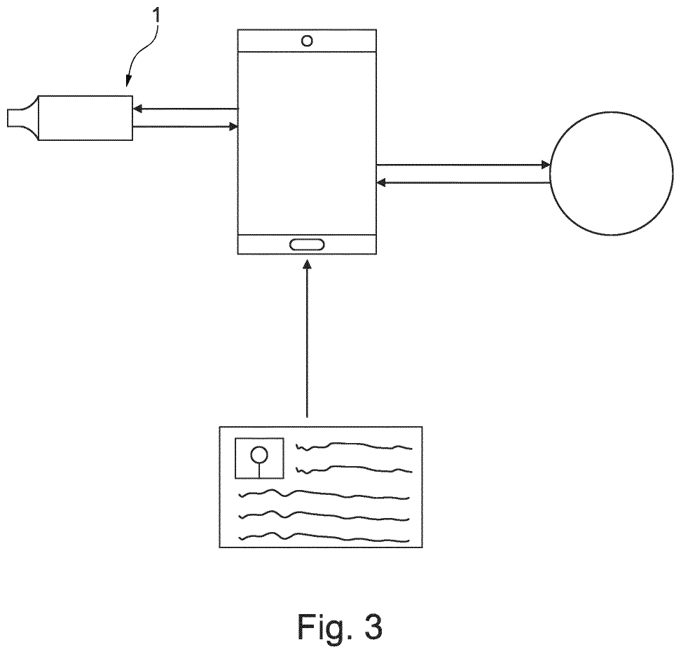

FIG. 3 verifying of a condition regarding an identity document

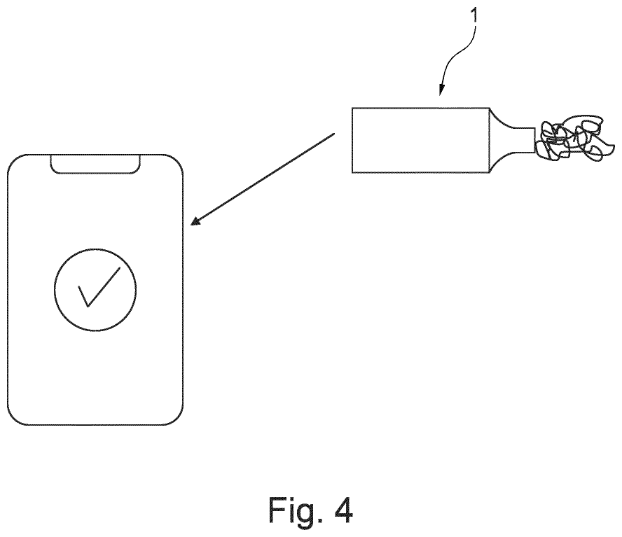

FIG. 4 displaying the unlocking state

FIGS. 5-8 a liquid container comprising restriction elements

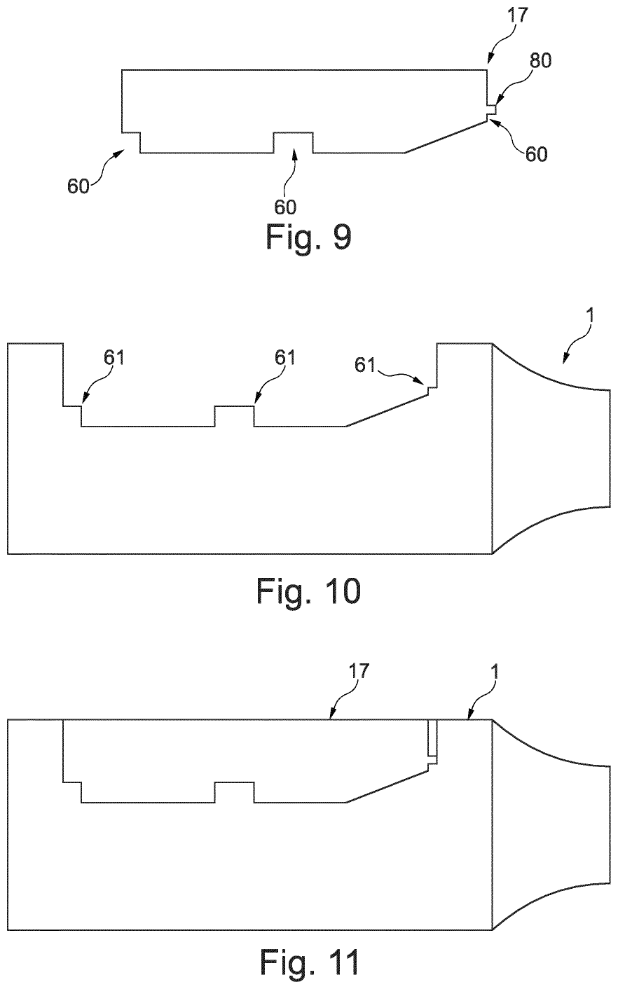

FIG. 9 a liquid container comprising restriction elements

FIG. 10 the mobile inhaler with delivering restrictions

FIG. 11 the mobile inhaler with delivering restrictions with a respective liquid container with restriction elements

In all figures apart from FIGS. 5 to 8, air intakes, corresponding valves etc. are not shown.

FIG. 1 shows an embodiment of the system comprising a mobile inhaler 1, a mouth piece 54 and a canal 55. The mobile inhaler is configured to accommodate one liquid container 17 that comprises two reservoirs that each comprise one component 11, 12 of the inhaling substance 10, which in this example consists in those two components 11, 12. All described features can nevertheless be applied to every system that was disclosed in the numbered embodiments, especially in case that the inhaling substance comprises more than 2 components.

The mobile inhaler 1 is configured to deliver the inhaling substance 10, which comprises at least one of the two components 11, 12. In this example, the system 1 delivers the first component 11 of the inhaling substance 10 only after verifying a condition, such as an age of a user of the mobile inhaler 1. Until the age of the user is not provided or if the age does not satisfy the verified condition, such as the condition being that the user is not underage, the system delivers in this example only the second component 12 of the inhaling substance 10, which is substantially free from nicotine or other psychoactive substances. FIG. 1 shows the first 11 and the second component 12 of the inhaling substance 10 in the canal 55 leading to the mouth piece 54.

FIG. 2 shows an embodiment of the system, comprising a mobile inhaler 1, a mouth piece 54, a canal 55, aerosol generators, two liquid containers 17 comprising each one component 11, 12 of the inhaling substance, connectors 80 configured for connection of the mobile inhaler 1 to the liquid containers 17 and a computing component 2. Furthermore, each of the liquid containers 17 comprises one restriction element 60 comprising the liquid containers' shapes, and the mobile inhaler 1 comprises two delivering restrictions 61 limiting the shape of attachable liquid containers 17.

In the embodiment shown in FIG. 2, the inhaling substance 10 that the system comprises, comprises two components 11, 12. It will be obvious to a person skilled in the art that the discussed considerations also apply to systems with more than two components of the inhaling substance 10.

The first liquid container 17 comprises a reservoir that comprises the first component 11 of the inhaling substance 10 that comprises nicotine in this example. Independently from the comprised nicotine, the first liquid container 17 comprises a restriction element 60 that comprises the liquid container's 17 shape. In this embodiment, the liquid container comprises a spherical cut-out. The liquid container 17 could nevertheless also comprise any other restriction element 60 as detailed above. The mobile inhaler 1 comprises a corresponding delivering restriction 61—in this embodiment a spherical counterpart. The liquid container 17 that comprises the second component 12 also comprises a restriction element 60 and the mobile inhaler 1 comprises a matching delivering restriction 61, wherein this restriction element 60 comprises a chamfer and the delivering restriction 61 comprises a matching protrusion. The delivering restrictions 61 of the mobile inhaler 1 can furthermore comprise elements for wireless or wired communication to a restriction element 60 comprised by one liquid container 17 that is an electronic element configured for at least an identification of the liquid container 17. This element can furthermore identify the component 11, 12 of the inhaling substance 10 comprised by the liquid container 17. One restriction element 60 of the left liquid container 17 can be for example an RFID chip that identifies the left container as a container comprising nicotine. One restriction element 60 of the right liquid container 17 can be an RFID chip identifying the liquid container 17 as a liquid container that does not comprise nicotine. Instead, the restriction elements 60 could also be different from each other, and they could furthermore be any other electronic elements configured at least for an identification of the corresponding liquid container 17. The mobile inhaler 1 can furthermore comprise delivering restrictions 61 that link different restriction elements 60, wherein this link may optionally be depending on the liquid container 17 or its content. An example would be a rule requiring liquid containers 17 that comprise an RFID-chip as restriction element 60 that provides the information that the liquid container 17 comprises nicotine to furthermore comprise a chamfer as restriction element 60. The mobile inhaler 1 would in this case for example comprise a device configured to retrieve information from the RFID-chip. The mobile inhaler 1 would furthermore comprise a switch that is configured to detect a chamfer or its absence at a mounted liquid container 17.

The person skilled in the art will understand that this linking delivering restrictions is not limited to nicotine as portion of a component comprised by a liquid container 17, nor that it is limited to the content of a liquid container 17 at all. It can furthermore also be applied to a system comprising only one liquid container 17 with two or more reservoirs. Also, in some embodiments, for the same contents, the restriction elements 60 could be different. In such embodiments, the mobile inhaler 1 can comprise delivering restrictions 61 that are matched by different restriction elements 60, e.g. by different data communicated by RFID-chips as restriction elements.

FIG. 3 shows an embodiment of the invention that comprises verifying a condition relating to an identity document that has to satisfy a further condition, in this case it has to provide an age of a user that is above a certain threshold and to provide at least one security feature. Verifying the condition is performed partially by an end user computer device that is connected to the mobile inhaler 1. In this case, the end user computer device is a smart phone. The end user computer device can for example take several images of the identity document. The mobile inhaler 1 is furthermore indirectly connected to a server via an end user computer device, that is in this embodiment said smart phone. The server receives the pictures of the identity document from the smart phone and verifies the security features of the identity document and calculates an age of an identity document's holder. The result is transferred to the end user computer device and from the end user computer device to the mobile inhaler 1. The person skilled in the art will understand that the present invention is not limited to the use of a smartphone as end user computer device. Furthermore, the invention is not limited to the use of visual pictures for the verification of documents, but that also wired communication to a chip mounted to the identity document or a wireless communication to an adapted device mounted to the identity document could be used to transfer data from the respective identity document to the end user computer device.

FIG. 4 shows an embodiment of the invention that comprises displaying a result of the verifying the set of conditions on a device linked to the mobile inhaler 1 before delivering the first component 11 of the inhaling substance 10. In the shown example, the device linked to the mobile inhaler 1 receives and displays data indicating that the verifying of the set of conditions led to a positive result and that therefore, the delivery of the first component 11 of the inhaling substance 10 can be performed.

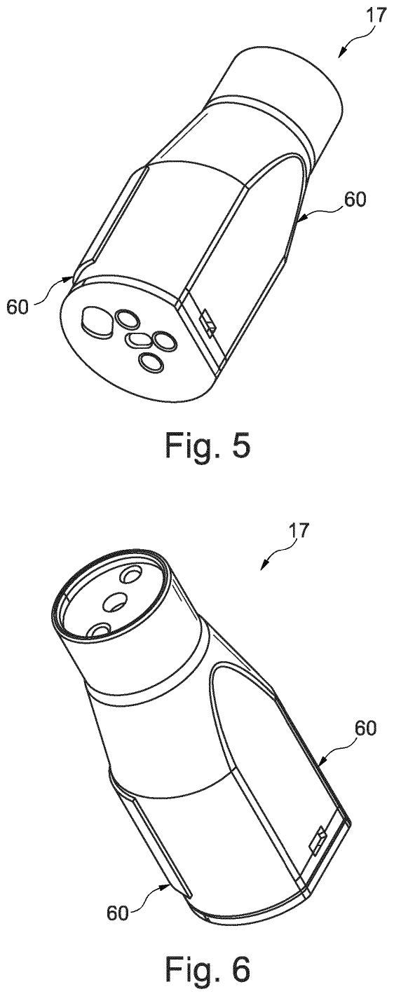

FIGS. 5 to 8 show embodiments of a liquid container 17. FIGS. 5 and 6 show an embodiment of the liquid container 17 comprising two restriction elements. One restriction element 60 comprises the shape of the liquid container 17. Another restriction element 60 comprises an electronic element configured to provide at least an identity of the liquid container 17, in this embodiment an RFID-chip that is attached to the liquid container, in FIG. 5 on the left side of the liquid container 17.

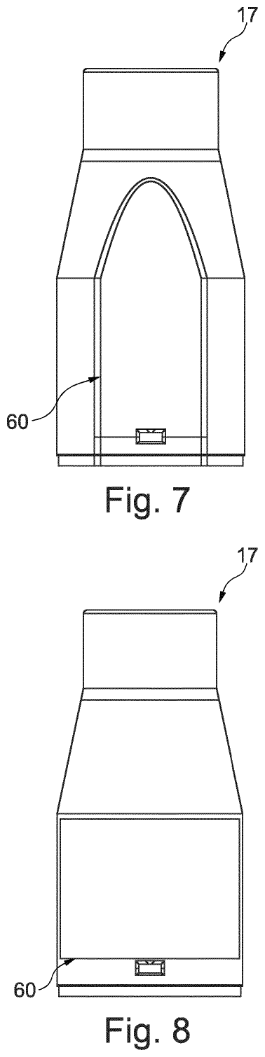

FIG. 7 shows the same embodiment of the liquid container 17 and the restriction element 60 that comprises the geometry of the liquid container 17.

FIG. 8 shows the same embodiment of a liquid container 17 and the restriction element 60 that comprises the RFID-chip.

FIGS. 9 to 11 show an embodiment of a liquid container 17 comprising restriction elements 60 and a connector 80, an embodiment of a mobile inhaler 1 comprising delivering restrictions 61 that are matched by these restriction elements 60 and an embodiment of the system comprising the embodiments of the liquid container 17 and the mobile inhaler 1.

In FIG. 9, the restriction elements 60 comprise the liquid container's 17 geometry, in particular, they comprise three indentations, one on the right of the liquid container's 17 connector 80, one in the middle and one on the left of the liquid container 17. The person skilled in the art will understand that the concrete geometry of the restriction elements 60 is not limiting and that furthermore, the liquid container 17 can comprise one or more reservoirs and one or more components 11, 12 of the inhaling substance 10.

FIG. 10 shows the mobile inhaler 1 comprising delivering restrictions 61 comprising each at least one protruding corner that is matched by the indentations of the liquid container 17. Chamfers and necessary tolerances in order to mount the liquid container 17 to the mobile inhaler 1 as well as the mobile inhaler's connector 80 are not shown.

FIG. 11 shows the system comprising the liquid container 17 and the mobile inhaler 1, wherein the liquid container 17 is mounted to the mobile inhaler 1. The delivering restrictions 61 match the restriction elements 60 according to the FIGS. 9 and 10.

The person skilled in the art will understand that neither the concrete shape of the delivering restrictions 61 and the restriction elements 60, nor the choice of restriction elements 60 and delivering restrictions 61 that only comprise the geometry of the mobile inhaler 1 and the liquid container 17 are limiting the invention.

The person skilled in the art will furthermore understand that the dosing arrangement(s), the aerosol generator(s) or parts or portions of any of the aforementioned can be a part of the mobile inhaler 1 and/or of at least one of the at least one or the plurality of liquid containers 17. That is, for the example of the aerosol generator, at least one liquid container 17 can comprise the aerosol generator, the mobile inhaler can comprise the aerosol generator or the mobile inhaler 1 can comprise a portion of the aerosol generator and at least one liquid container 17 can comprise a portion of the aerosol generator. The person skilled in the art will understand that the mobile inhaler 1 and the at least one of the at least one or the plurality of liquid containers 17 are chosen as examples for portions of the system.

1-16. (canceled)

17. A system comprising
a mobile inhaler that is configured to be simultaneously connected to at least one or a plurality of liquid containers, and to deliver an inhaling substance, wherein the inhaling substance comprises at least one of an amount of a first component and an amount of a second component,
wherein the mobile inhaler further comprises
at least one or a plurality of connector(s) configured for connection of the mobile inhaler
to the at least one or the plurality of liquid containers and for intaking of at least a portion of each of the liquid container(s)'s content(s);
wherein the system comprises furthermore
the at least one or the plurality of liquid containers, wherein the at least one or the plurality of liquid containers comprises a plurality of reservoirs, wherein each reservoir comprises a component of the inhaling substance; and
wherein the reservoirs each comprise mutually different components of the inhaling substance.

18. The system according to claim 17,
wherein the mobile inhaler comprises a connection component to an output interface component,
wherein the output interface component is configured to display information relating to the mobile inhaler, a delivery of the inhaling substance and/or an inhalation, information relating to a recording and/or a duration of an inhalation, a summary or an analysis thereof or information relating to at least one liquid container that is, was or can be connected to the mobile inhaler.

19. The system according to claim 17,
wherein the system comprises furthermore an interface suitable to connect the system to at least one of a computer device, an integrated circuit and a data storage device, by at least one of a wired and a wireless connection.

20. A method for operating a system according to claim 17, comprising
delivering the inhaling substance by the system,
wherein the system delivers at least the first component of the inhaling substance according to a set of conditions, and
wherein said set of conditions regulates at least one of a quantity and a general delivery of at least one of the first component and the inhaling substance.

21. The method according to claim 20,
comprising at least partially automatically regulating at least the amount the first component of the inhaling substance that is delivered over time.

22. The method according to claim 20,
wherein automatically regulating the amount of the first component that is delivered over time follows at least one of
(a) a pre-defined pattern or function,
(b) a pre-defined rule-set,
(c) a pattern based on data that is at least one of measured, generated and recorded by the system or the mobile inhaler,
(d) a mathematical model or function based on data that is at least measured, generated and/or recorded by the system or the mobile inhaler,
(e) a machine learning or artificial intelligence model; and
(f) further data.

23. The method according to claim 20,
comprising furthermore verifying a set of conditions before delivering the first component of the inhaling substance.

24. The method according to the claim 23,
wherein the step of delivering of the first component of the inhaling substance before which said set of conditions is verified is the first delivery of the first component of the inhaling substance.

25. The method according to claim 23,
wherein said set of conditions comprises a condition concerning an age of a person.

26. The method according to claim 23,
comprising furthermore
(a) verifying at least one condition of the set of conditions at least partially by an end user computer device that is connected to the mobile inhaler or to the system;
(b) verifying at least one condition of the set of conditions at least partially by a server that is connected to the mobile inhaler or to the system;
(c) verifying at least one of the at least one or the set of condition(s) at least partially by a server that is at least indirectly connected to the mobile inhaler via an end user computer device that is connected to the mobile inhaler;
(d) verifying at least one condition of the set of conditions at least partially by a computing component that the system comprises; or
(e) combining any of the verifying steps according to the preceding four options.

27. The method according to claim 23,
comprising furthermore displaying at least an indicator for said verifying or its result on at least one of a portion of the system, the mobile inhaler and a device linked to the system via a wireless or wired connection.

28. The method according to claim 20,
comprising furthermore delivering at least one of the first component and the second component of the inhaling substance only from liquid container(s) that comprise(s) at least one restriction element, and/or if there is another authorization.

29. The method according to claim 20,
comprising furthermore counting at least one number of inhales since a point in time and storing the at least one the number of inhales on at least one of the system, a portion thereof, such as an electronic element that one of the at least one or the plurality of liquid containers comprises, and a third device, such as a smart handheld device, a mobile computer or a dongle,
wherein this third device is configured for wired and/or wireless communication.

30. The method according to claim 20,
comprising storing an unlocking state,
wherein the unlocking state is a result of the step of verifying the set of conditions or an indicator for this result.
